(19) United States
(12) Patent Application Publication
(10) Pub. No.: US 2025/0134770 A1
(43) Pub. Date: May 1, 2025

(54) PRE-FILLED MULTI-FLUID MEDICAL DELIVERY ASSEMBLIES

(71) Applicant: Kosaka Family Limited, East Sussex (GB)

(72) Inventors: Jeff PRICE, Windermere, FL (US); Daniel AMENT, Denver, CO (US); Jordan Michael YOST, Erie, PA (US); Jason Christopher BRYANT, Erie, PA (US)

(21) Appl. No.: 19/003,087

(22) Filed: Dec. 27, 2024

Related U.S. Application Data
(63) Continuation of application No. PCT/US24/48028, filed on Sep. 23, 2024.
(60) Provisional application No. 63/539,918, filed on Sep. 22, 2023.

(51) Int. Cl. A61J 1/14

(52) U.S. Cl. CPC A61J 1/1406

(57) ABSTRACT

Disclosed herein are pre-filled multi-fluid medical delivery assemblies and methods for fabrication and use thereof.

### CROSS-REFERENCE TO RELATED APPLICATIONS

This application is a continuation of PCT Application Serial No. PCT/US24/48028, filed Sep. 23, 2024, and titled “PRE-FILLED MULTI-FLUID MEDICAL DELIVERY ASSEMBLIES”, which claims the benefit and priority under 35 U.S.C. § 119 (e) to, U.S. Provisional Patent Application No. 63/539,918 filed on Sep. 22, 2023 and titled “PRE-FILLED MULTI-FLUID MEDICAL DELIVERY ASSEMBLIES”, the entire contents of each disclosure being incorporated by reference herein.

### STATEMENT REGARDING FEDERALLY SPONSORED RESEARCH

This invention was made with government support under Award No. 75A50120C00007, awarded by the Department of Health and Human Services (HHS). The government has certain rights in the invention.

### BACKGROUND

Disposable injector devices for administrating a medical agent, such as a vaccine, medicament, drug, etc., typically include a single chamber for storing the medical agent which is distributed to the patient through a needle of the injector device. However, some medical agents may include multiple medicaments which require independent storage (and sometimes subsequent mixing) prior to delivery to the subject as a single dose. Dual chamber injection devices for separately storing multiple liquid agents until time of administration and/or for combined delivery of the agents to the subject are known, but are complex, not cost effective to produce, and/or not feasible to manufacture in large quantities and/or in a short amount of time if demand unexpectedly peaks.

In addition, some fluid agents stored in such devices have a defined shelf life. Because such devices are not able to be produced in large quantities at scale to meet unexpected peak demand, providers of such devices are put in a position of paying to pre-order and have manufactured inventories of such devices that sometimes need to be discarded due to expiration dates. Not only are these devices not cost-effective to manufacture in the first place, but they are also subject to waste.

Previous attempts at providing single-use or disposable injection devices to remedy such problems in the industry have failed to adequately remedy the existing problems.

### SUMMARY

In accordance with one illustrative embodiment of the present invention, a pre-filled medical delivery assembly comprises a first blow-fill-seal (BFS) module having a first fluid seal and a first reservoir with a first fluid agent therein, a second BFS module having a second fluid seal and a second reservoir with a second fluid agent therein, a barrel member defining first and second recesses for receiving therein the first and second BFS modules, respectively, a connector assembly having first and second piercing regions where each piercing region has a piercing member therein and with the first piercing region being disposed with respect to the first recess of the barrel member and the second piercing region being disposed with respect to the second recess of the barrel member, an administration assembly comprising an administration conduit with the administration assembly being coupled to the connector assembly such that the administration conduit is in fluid communication with each of the first and second piercing regions and a plunger configured to move axially with respect to the barrel member with the plunger having first and second actuation portions disposed within the barrel member and constructed to engage with the first and second BFS modules, respectively. The pre-filled medical delivery assembly is configured such that axial movement of the plunger relative to the administration assembly causes the piercing members of the first and second piercing regions to penetrate the first and second fluid seals, respectively to permit distribution of the first fluid agent and the second fluid agent through the administration conduit.

In embodiments, a volume of the first reservoir is different than a volume of the second reservoir, and a volume of the first fluid agent is different than a volume of the second fluid agent. In some embodiments, a volume of the second reservoir is at least two (2) times greater than a volume of the first reservoir, and a volume of the second fluid agent is at least two (2) times greater than a volume of the first fluid agent. In other embodiments, a volume of the second fluid agent is about 2 ml, and a volume of the first fluid agent is about 0.7 ml.

In embodiments, at least one of the first fluid agent or the second fluid agent comprises one of a vaccine, a drug, a medicament, or a component of any of the foregoing.

In some embodiments, at least one of the first fluid seal or the second fluid seal comprises a foil, wax, paper, a reduced-thickness section of the respective BFS module, or any combination of the foregoing.

In other embodiments, the administration conduit comprises a needle or cannula. In certain embodiments, the needle has a length in a range of 0.5 mm to 4 mm, inclusive, or in a range of 4 mm to 15 mm, inclusive, or in a range of 15 mm to 30 mm, inclusive.

In embodiments, an outlet of the administration conduit is formed as a nozzle configured to facilitate dispersion of a combination of the first and second fluid agents into a spray or one or more droplets.

In some embodiments, one, some, or all of the barrel member, the connector assembly, and the plunger are formed of a respective material having a hardness greater than that of the first BFS module, the second BFS module, or both the first and second BFS modules.

In other embodiments, the connector assembly and the administration assembly are coupled together so as to define an internal conduit between the connector assembly and the administration assembly, and the internal conduit fluidically connects the first and second reservoirs to the administration conduit via the respective first and second piercing regions. In some embodiments, the internal conduit has a first section adjacent to an outlet from the first piercing region, a second section adjacent to an outlet from the second piercing region, and a third section adjacent to an inlet of the administration conduit and wherein the third section is disposed between the first and second sections. In certain embodiments, the third section is disposed at a midpoint between the first and second sections. In yet other embodiments, the third section is disposed between the first and second sections with respect to a radial direction of the administration conduit. In other embodiments, the third section is disposed between the first section and the first piercing region and/or between the second section and the second piercing region with respect to an axial direction of the administration conduit.

In embodiments, each piercing member comprises a needle.

In other embodiments, the barrel member comprises an axially-extending internal partition that divides the first recess from the second recess. In certain embodiments, at least part of the first actuation portion is tapered so as to compress the first reservoir against the internal partition as the plunger moves axially toward the administration assembly, and/or at least part of the second actuation portion is tapered so as to compress the second reservoir against the internal partition as the plunger moves axially toward the administration assembly.

In some embodiments, the barrel member comprises a pair of lateral sections coupled together.

In other embodiments, the first BFS module comprises a first neck portion with the first seal at an end thereof, and the second BFS module comprises a second neck portion with the second seal at an end thereof. In certain embodiments, the first neck portion comprises a first mating feature with the first piercing region comprising a first cooperating feature configured to engage with the first mating feature. The second neck portion comprises a second mating feature with the second piercing region comprising a second cooperating feature configured to engage with the second mating feature. In certain embodiments, the first mating feature comprises a first laterally-protruding portion and the second mating feature comprises a second laterally-protruding portion. In certain embodiments, the first mating feature has a toroidal shape in a side view, and the first cooperating feature comprises a scalloped portion of a sidewall of the first piercing region and the second mating feature has a toroidal shape in a side view, and the second cooperating feature comprises a scalloped portion of a sidewall of the second piercing region.

In embodiments, the first BFS module and the second BFS module each comprises one or more flanges constructed to engage with one of the barrel member or the connector assembly.

In other embodiments, a first longitudinal direction of the first BFS module extends from the first reservoir toward the first fluid seal and a second longitudinal direction of the second BFS module extends from the second reservoir toward the second fluid seal. The first and second longitudinal directions are substantially parallel to and spaced apart from each other along a radial direction. In certain embodiments, the first and second longitudinal directions are on opposite sides of the administration conduit.

In embodiments, at least part of the piercing members of the first and second piercing regions overlap with at least part of the administration conduit.

In some embodiments, the first piercing region and the second piercing region are staggered wherein a first piercing end of the first piercing region is longitudinally displaced relative to a second piercing end of the second piercing region.

In another illustrative embodiment of the present invention, a pre-filled medical delivery assembly comprises a first blow-fill-seal (BFS) module having a first fluid seal and a first reservoir with a first fluid agent therein, a second BFS module having a second fluid seal and a second reservoir with a second fluid agent therein, a barrel member for receiving therein the first and second BFS modules, and defining a longitudinal axis, a connector assembly having first and second piercing regions with the first piercing region having a first piercing end and the second piercing region having a second piercing end displaced longitudinally with respect to the first piercing end of the first piercing region, an administration assembly comprising an administration conduit with the administration assembly being coupled to the connector assembly such that the administration conduit is in fluid communication with each of the first and second piercing regions and a plunger constructed to move axially with respect to the barrel member, to engage the first and second BFS modules to cause the piercing members of the first and second piercing regions to penetrate, in succession, the first and second fluid seals, respectively.

In some embodiments, the barrel member defines first and second recesses for receiving therein the first and second BFS modules, respectively.

In accordance with another illustrative embodiment, a method comprises introducing a blow fill seal component into a barrel component where the blow fill seal component includes a first blow-fill-seal (BFS) module having a first fluid seal and a first reservoir with a first fluid agent therein and a second BFS module having a second fluid seal and a second reservoir with a second fluid agent therein, at least partially positioning a plunger component into the barrel component and advancing the plunger component within the barrel component along an axis of the barrel component to cause a first internal penetration member associated with the barrel component to penetrate the first fluid seal of the first BFS module and a second internal penetration member associated with the barrel component to penetrate the second fluid seal of the second BFS module, to cause release of the first fluid agent and the second fluid agent for distribution through an administration component coupled relative to the barrel component.

In certain embodiments, the method includes coupling the administration component relative to the barrel component.

In other embodiments, the method further includes mixing the first fluid agent and the second fluid agent within a conduit in fluid communication with the administration component.

In some embodiments, introducing a blow seal component includes at least partially positioning the first BFS module into a first barrel recess of the barrel component and the second BFS module into a second barrel recess of the barrel component.

In embodiments, the method includes coupling a connector component to the barrel component with the connector component including the first and second internal penetration members and the conduit.

In other embodiments, the first internal penetration member is axially displaced relative to the second internal penetration member, wherein advancing the plunger component includes the first internal penetration member penetrating the first fluid seal prior to the second internal penetration member penetrating the second fluid seal.

In another illustrative embodiment, a pre-filled medical delivery assembly comprises a blow-fill-seal (BFS) vial including a first vial module having a first fluid seal and a first reservoir with a first fluid agent therein and a second vial module having a second fluid seal and a second reservoir with a second fluid agent therein, and a delivery module couplable to the BFS vial. The delivery module includes a main body for at least partially receiving the BFS vial, a first internal penetrating member disposed within the main body and defining a first fluid conduit, a second internal penetrating member disposed within the main body and defining a second fluid conduit, and an administration member mounted to the delivery module. Upon coupling of the BFS vial and the delivery module, the first internal penetrating member penetrates the first fluid seal of the first vial module and the second internal penetrating member penetrates the second fluid seal of the second vial module. The administration member is in fluid communication with the first fluid conduit of the first internal penetrating member and in fluid communication with the second fluid conduit of the second internal penetrating member to enable delivery of the first and second fluid agents through the administration member.

In embodiments, the first internal penetrating member is axially aligned with the first fluid seal of the first vial module and the second internal penetrating member is axially aligned with the second fluid seal of the second vial module upon coupling of the BFS vial and the delivery module.

In some embodiments, the main body includes a pair of locking arms with locking detents. The locking detents are received within cooperating locking recesses of the BFS vial to secure the BFS vial to the delivery module.

In other embodiments, the administration member includes an administration hub. The administration hub is coupled to the main body of the delivery module. The administration hub defines at least part of a mixing chamber for receiving the first and second fluid agents for delivery through the administration member.

In embodiments, the main module includes a hub connector for coupling to the administration hub.

In some embodiments, the first and second internal penetrating members are integrally formed with the hub connector.

In other embodiments, the first and second internal penetrating members are staggered whereby the first internal penetrating member and the second internal penetrating member penetrate the respective first and second fluid seals in succession.

In embodiments, the first internal penetrating member defines a length greater than a corresponding length of the second internal penetrating member.

In some embodiments, a removable protective cover is disposed over the administration member.

In other embodiments, a pivoting shield is pivotally mounted relative to the main body of the delivery module. The pivoting shield is pivotal to selectively cover and expose the administration member.

### BRIEF DESCRIPTION OF THE DRAWINGS

The figures depict embodiments for purposes of illustration only. The accompanying figures are not necessarily drawn to scale, emphasis instead being placed upon illustrating the principles disclosed herein. The figures are included to further the understanding of the various aspects and embodiments and are incorporated in and constitute a part of this specification, but are not intended as a definition of the limits of any particular embodiment. In the figures, each identical or nearly identical component that is illustrated in various figures is represented by a like numeral. For purposes of clarity, not every component may be labeled in every figure. One skilled in the art will readily recognize from the following description that alternative embodiments of the examples illustrated herein may be employed without departing from the principles described herein, wherein:

FIG. 1 is a simplified schematic diagram illustrating component and aspects of assembly and use of a pre-filled multi-fluid medical delivery assembly, according to one or more embodiments of the subject disclosure;

FIGS. 2A-2E show perspective, side, top, cross-sectional, and magnified cross-sectional views, respectively, of another pre-filled multi-fluid medical delivery assembly, according to one or more embodiments of the subject disclosure;

FIGS. 3A-3D show perspective, top, front, and side views, respectively, of a first BFS module for use in a pre-filled multi-fluid medical delivery assembly, according to one or more embodiments of the subject disclosure;

FIGS. 4A-4D show perspective, top, front, and side views, respectively, of a second BFS module for use in a pre-filled multi-fluid medical delivery assembly, according to one or more embodiments of the subject disclosure;

FIGS. 5A-5D show perspective, top, cross-sectional, and side views, respectively, of a connector assembly of a pre-filled multi-fluid medical delivery assembly, according to one or more embodiments of the subject disclosure;

FIGS. 6A-6D show perspective, top, side, and cross-sectional views, respectively, of an administration assembly of a pre-filled multi-fluid medical delivery assembly, according to one or more embodiments of the subject disclosure;

FIGS. 7A-7E show perspective, top, internal side, bottom, and front views, respectively, of a first barrel section of a pre-filled multi-fluid medical delivery assembly, according to one or more embodiments of the subject disclosure;

FIG. 7F is a magnified view of a connection feature of the first barrel section of FIGS. 7A-7E;

FIGS. 8A-8E show perspective, top, internal side, internal bottom, and front views, respectively, of a second barrel section of a pre-filled multi-fluid medical delivery assembly, according to one or more embodiments of the subject disclosure;

FIG. 8F is a magnified view of a connection feature of the second barrel section of FIGS. 8A-8E;

FIGS. 9A-9D show perspective, top, front, and side views, respectively, of a plunger of pre-filled multi-fluid medical delivery assembly, according to one or more embodiments of the subject disclosure;

FIGS. 10A-10C are sequential cross-sectional views illustrating actuation of the pre-filled multi-fluid medical delivery assembly;

FIGS. 11A-11C show perspective, side elevation and top views of another pre-filled multi-fluid medical delivery assembly, according to one or more embodiments of the subject disclosure;

FIG. 12 is an exploded perspective view of the pre-filled multi-fluid medical delivery assembly of FIGS. 11A-11C, according to one or more embodiments of the subject disclosure;

FIG. 13 is a partial cross-sectional view of the pre-filled multi-fluid medical delivery assembly of FIGS. 11A-12, according to one or more embodiments of the subject disclosure;

FIGS. 14A-14C show perspective, side elevation and top views of another pre-filled multi-fluid medical delivery assembly, according to one or more embodiments of the subject disclosure;

FIG. 15 is an exploded perspective view of the pre-filled multi-fluid medical delivery assembly of FIGS. 14A-14C, according to one or more embodiments of the subject disclosure;

FIGS. 16 and 17 are partial side elevation views of BFS vial of a pre-filled multi-fluid medical delivery assembly of FIGS. 14A-15, according to one or more embodiments of the subject disclosure;

FIG. 18 is a partial side elevation view of the pre-filled multi-fluid medical delivery assembly of FIGS. 14A-17, according to one or more embodiments of the subject disclosure;

FIG. 19 is a partial cross-sectional elevation view of the pre-filled multi-fluid medical delivery assembly of FIGS. 14A-18, according to one or more embodiments of the subject disclosure; and

FIG. 20 is a partial view of an internal penetrating member of a pre-filled multi-fluid medical delivery assembly, according to one or more embodiments of the subject disclosure.

### DETAILED DESCRIPTION

### I. Introduction

Described herein are systems, assemblies, kits, and methods for medical delivery of multiple fluid (e.g., liquid) agents (e.g., at least two) to a patient (e.g., human or animal) from pre-filled (and, in some embodiments, field-assembled or assembled at the point-of-use) modules (also referred to herein as a cartridge, bottle, or vial). In some embodiments, at least two fluid agents are sealed in separate modules and maintained separate from each other until a time when it is desirable to administer and/or mix the fluid agents (e.g., at the time of or prior to use and/or administration to the patient). The fluid agents can be any type of agent to be injected into or otherwise delivered to a patient and capable of producing a therapeutic effect, either alone or in combination with an active ingredient. Accordingly, the fluid agents can include, but are not limited to, separate vaccines, drugs, medicaments, adjuvants, diluents, active ingredients, etc. that are desirable to combine for use and/or common administration to the patient. For example, in some embodiments, the combination of the fluid agents can form a multi-fluid agent, solution, mixture, suspension, etc. Alternatively or additionally, the fluid agents can be separate components that, when combined, form a vaccine, drug, medicament, etc. For example, in some embodiments, the combination of the fluid agents can comprise a single dose of a therapeutic agent (e.g., vaccine, drug, medicament, etc.). In some embodiments, one or more of the fluid agents in each module can be tracked, monitored, checked for compatibility, etc., such as by utilization of electronic data storage devices (not shown) coupled to the various modules or components of the system.

In some embodiments, each module may comprise a blow-fill-seal (BFS) module that has at least one reservoir (also referred to herein as chamber) prefilled with one or more fluid agents using a BFS manufacturing technique. In some embodiments, the BFS module can have multiple reservoirs, and at least some of the reservoirs filled with fluid agents can be sealed from other of the reservoirs, thereby maintaining the fluid agents separate until combination thereof is desired (e.g., a time for use and/or administration to the patient). The BFS module may be constructed, filled, and sealed, according to some embodiments, in a sterile manufacturing environment. BFS modules may, for example, offer a less expensive alternative to typical vials or bottles created via other manufacturing techniques. In some embodiments, BFS modules (e.g., due to the nature of the BFS manufacturing process) may not require separate sterilization (e.g., and may accordingly be compatible with a wider array of fluid agents), may provide enhanced production rates of sterile/aseptic units per hour, and/or may be provided to an end-user for significantly lower per dose/unit costs. In some embodiments, these advantages may come with attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989), which is incorporated herein by reference.

In some embodiments, a connector assembly (also referred to herein as a manifold, coupling, or fluid sub-assembly) can be coupled to a BFS module to breach a fluid seal thereof, thereby allowing fluidic access to the reservoir (e.g., fluid agent can be added to and/or removed from the reservoir) of the BFS module. In some embodiments, the fluid seal of the BFS module can comprise a foil, wax, paper, a section of the BFS module (e.g., a thinned section or wall of the BFS module), or any combination of the foregoing. In some embodiments, the fluid seals can be breached, for example, by puncturing, piercing, rupturing, penetrating, or otherwise breaking the seal, in order to access and/or dispense contents of the fluid reservoirs.

In some embodiments, the BFS module can have a neck portion that has a laterally-protruding portion that interfaces with a mating feature in a piercing region (also referred to herein as recess, bore, port, chamber, or opening) of the connector assembly when the neck portion is inserted therein, which can facilitate coupling of the BFS module to the connector assembly. In some embodiments, an administration assembly can be coupled to the connector assembly to form an internal fluid conduit, within which the fluid agent from one BFS module can be combined with the fluid agent from another BFS module prior to use and/or administration. For example, the internal fluid conduit can be formed between facing surfaces of the connector assembly and the administration assembly.

In some embodiments, the combined fluid agents can be administered to a patient via the administration assembly. In some embodiments, a barrel member (also referred to herein as housing or shell) can be coupled to, or at least disposed adjacent to, the connector assembly, for example, on a side opposite from the administration assembly. At least part of the BFS modules can be contained within the barrel member. In some embodiments, an actuation member (e.g., plunger) can have a portion disposed within and axially movable with respect to the barrel member. In some embodiments, axial movement of the actuation member toward the administration assembly can push the BFS modules into engagement with piercing members of the connector assembly (e.g., by urging the neck portion of the BFS module from an interior of the barrel member into the respective piercing region). For example, the axial movement of the actuation member can cause the piercing members to breach and/or extend through the respective fluid seals of the BFS modules, thereby making a fluid connection between the reservoirs of the BFS module and the internal conduit and/or an administration conduit of the administration assembly. Alternatively or additionally, in some embodiments, the axial movement toward the administration assembly can at least partially compress (simultaneously or sequentially) part of the BFS modules (e.g., the respective reservoirs thereof) so as to cause the fluid agent therein to be dispensed into the internal conduit and subsequently from the administration conduit. Alternatively, in some embodiments, the BFS modules are coupled to the connector assembly such that the respective fluid seals are pierced, after which the barrel member with actuation member therein is placed around the exposed portions of the BFS modules. The connector assembly may thus be considered coupled to the barrel member via the BFS modules.

In some embodiments, the administration assembly comprises a needle or cannula constructed for subcutaneous, intramuscular, intradermal, or intravenous injection of the combined fluid agents into the patient. Alternatively, in some embodiments, the administration assembly comprises a nozzle. For example, the nozzle can be a spray nozzle that facilitates dispersion of the combined liquid agents into a spray, which configuration may be useful in the administration of the combined fluid agents into a body cavity or orifice (e.g., nasal passage, ear canal, etc.). In another example, the nozzle can be a droplet nozzle that facilitates formation of droplets of the combined fluid agents, which configuration may be useful in the administration of the combined fluid agents to the eyes, for topical application, etc.

In some embodiments, the administration assembly, the first connector, and/or the second connector may be configured to be coupled and/or assembled to the BFS modules on-site and/or in the field. Alternatively or additionally, in some embodiments, a connector may be coupled and/or assembled to a BFS module in a manufacturing facility and provided to users as a single, pre-assembled fluid sub-assembly (e.g., with a neck of the BFS module inserted into a recess of the connector, but without breach of the sealed port of the BFS module). In some embodiments, the pre-filled multi-fluid medical delivery assembly may, for example, be capable of delivering combined fluid agents in a controlled manner and without requiring specialized skill in assembling and/or administering delivery of such agents.

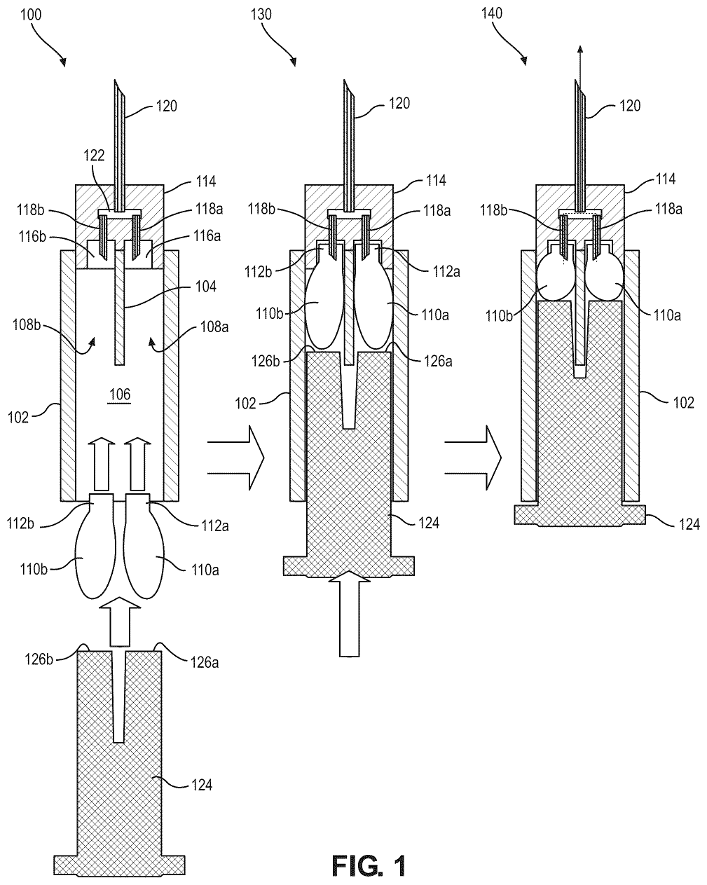

FIG. 1 illustrates components of a pre-filled multi-fluid medical delivery assembly and operation thereof. In the illustrated example, the medical delivery assembly includes a syringe barrel member 102, a first BFS module 110a, a second BFS module 110b, a connector assembly 114, an administration conduit 120, and an actuation member 124. The syringe barrel member 102 can have a barrel partition 104 that divides interior volume 106 into a first module recess 108a for the first BFS module 110a and a second module recess 108b for the second BFS module 110b. The connector assembly 114 can have a first piercing region 116a for receiving a first BFS module neck portion 112a of the first BFS module 110a, and a second piercing region 116b for receiving a second BFS module neck portion 112b of the second BFS module 110b. A terminal end of the first piercing region 116a can have a first piercing member 118a (e.g., needle, such as a 23-gauge stainless steel needle). Similarly, a terminal end of the second piercing region 116b can have a second piercing member 118b. In some embodiments, either or both of the first and second piercing regions 116a, 116b and/or the first and second piercing members 118a, 118b may be fabricated from a polymeric material to minimize cost. In embodiments, the first and second piercing members 118a, 118b may terminate at the same location within connector assembly 114. In other embodiments, the first and second piercing members 118a, 118b may be staggered. Staggering the first and second piercing members 118a, 118b may reduce force profiles required to penetrate the first and second BFS modules 110a, 110b. Staggering will cause the first and second piercing members 118a, 118b to pierce the first and second BFS modules 110a, 110b in succession. In the illustrated example, a conduit 122 is provided within or as part of the connector assembly 114 to fluidically connect administration conduit 120 (e.g., needle, such as a 23-gauge stainless steel needle) to respective outlets of the first piercing region 116a and second piercing region 116b. However, in some embodiments, conduit 122 could instead be formed by a space between the connector assembly 114 and an assembly of the administration conduit 120 (e.g., a needle hub) coupled to the connector assembly 114.

In an initial unassembled state 100, the actuation member 124 can be arranged outside of syringe barrel member 102. The first BFS module 110a can be loaded into interior volume 106 of the syringe barrel member 102, for example, the first module recess 108a on one side of barrel partition 104. Before, after, or during the loading of the first BFS module 110a, the second BFS module 110b can also be loaded into interior volume 106 of the syringe barrel member 102, for example, the second module recess 108b on an opposite side of barrel partition 104. In an assembled state 130, the actuation member 124 can be disposed within syringe barrel member 102, such that first actuation portion 126a of the actuation member 124 contacts or faces first BFS module 110a and such that second actuation portion 126b of the actuation member 124 contacts or faces second BFS module 110b. Axial movement of the actuation member 124 toward the administration conduit 120 and/or connector assembly 114 engages the actuation portions with the respective BFS modules, thereby pushing the first BFS module neck portion 112a into first piercing region 116a such that first piercing member 118a breaches the fluid seal of the first BFS module 110a and pushing the 112b into second piercing region 116b such that second piercing member 118b breaches the fluid seal of the second BFS module 110b. Together with conduit 122, the piercing members can provide a fluidic connection between the contents of the BFS modules and the administration conduit 120. In some embodiments, one or both of the piercing members 118a, 118b can be disposed or arranged within the respective piercing region 116a, 116b so as to be substantially aligned with a center of a facing area of the respective BFS module neck portion 112a, 112b (e.g., extending collinear with a central axis of the neck portion and/or, at the point of piercing, a tip of the piercing member is substantially at the center of the facing area). Alternatively, in some embodiments, one or both of the piercing members 118a, 118b can be disposed or arranged within the respective piercing region 116a, 116b so as to be offset from a center of a facing area of the respective BFS module neck portion 112a, 112b (e.g., extending substantially parallel to but offset from a central axis of the neck portion and/or, at the point of piercing, a tip of the piercing member is displaced from the center of the facing area). As shown in actuated state 140, further axial movement of the actuation member 124 toward administration conduit 120 and/or connector assembly 114 compresses the respective BFS modules, thereby causes the contents thereof to flow into the conduit 122 and/or administration conduit 120 for subsequent delivery to a patient. In some embodiments, the flow into the conduit 122 and/or administration conduit 120 may cause the liquids from the BFS modules to mix prior to or concurrent with delivery to the patient.

Alternatively, in some embodiments, the syringe barrel member 102 can be formed as separate interlocking halves. The first BFS module 110a and second BFS module 110b, as well as the first actuation portion 126a and second actuation portion 126b of the actuation member 124 can be disposed between the barrel member halves before the barrel member halves are coupled together. In such a configuration, the actuation member 124 can be retained by the barrel member 102 while still capable of axial movement with respect thereto, for example, to engage the BFS modules with the respective piercing member and then to compress the BFS modules to dispense the fluid contents therefrom.

### II. Pre-filled Dual-Liquid Medical Delivery Assembly

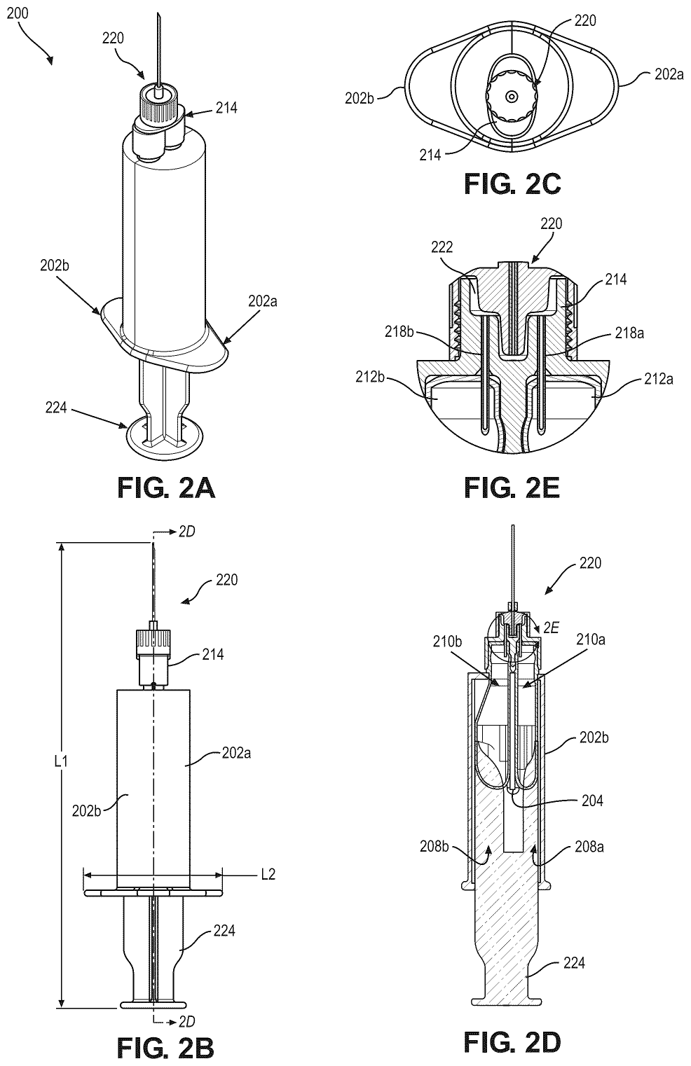

Referring to FIGS. 2A-10C, a pre-filled dual-liquid medical delivery assembly 200 can have a first barrel section 202a, a second barrel section 202b, a plunger 224, a connector assembly 214, and an administration assembly 220. The first barrel section 202a and second barrel section 202b can comprise separate halves that, when coupled together, form a housing enclosing an interior volume. At least an upper portion of the enclosed interior volume can be separated into a first module recess 208a and a second module recess 208b by a barrel partition 204 formed by one or both the barrel sections. The connector assembly 214 is coupled at an upper axial end of the housing formed by the coupled barrel sections, and the administration assembly 220 is coupled to the connector assembly 214 on an opposite axial side from the barrel sections. In embodiments, the connector assembly 214 may be integrally formed with the first barrel section 202a and second barrel section 202b or alternatively a separate component and connected via conventional methodologies including for example, a threaded coupling, bayonet coupling, locking ledges or the like. As shown in FIG. 2E, the coupling between the connector assembly 214 and the administration assembly 220 forms a gap that serves as a conduit 222 fluidically interconnecting a needle of the administration assembly 220 to the first piercing needle 218a and second piercing needle 218b of the connector assembly 214. At least a portion of plunger 224 can be disposed within the first and second barrel sections and can be constructed to move axially therein (e.g., toward or away from the connector assembly 214 and/or administration assembly 220).

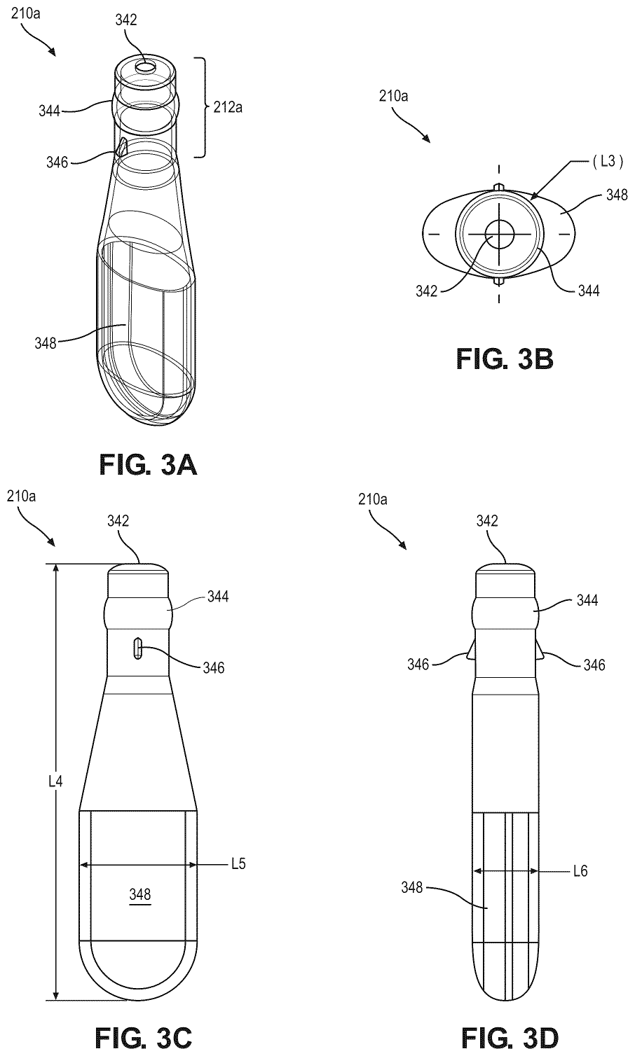

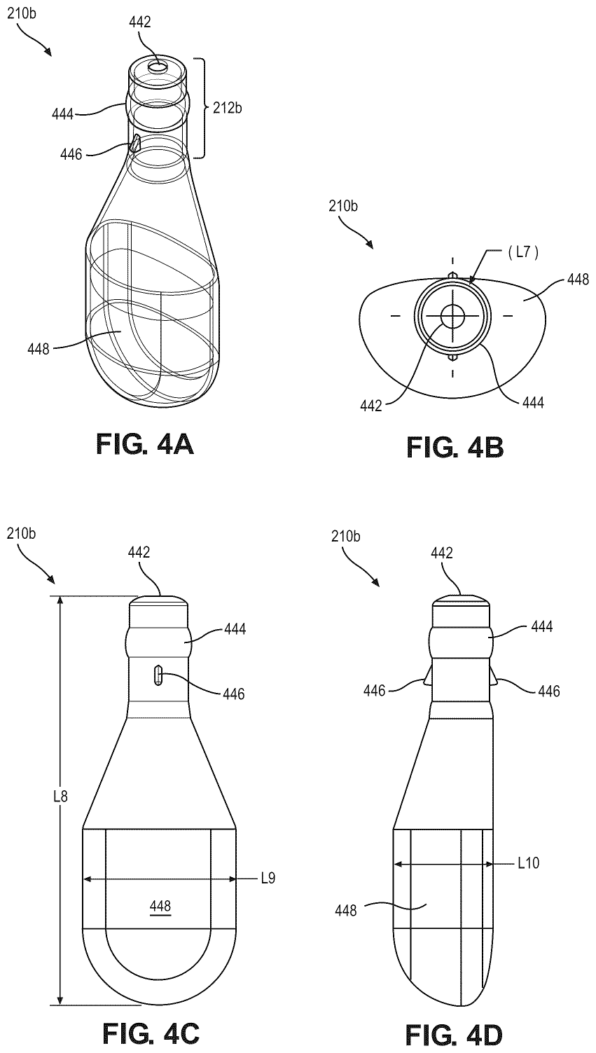

The pre-filled dual-liquid medical delivery assembly 200 can also have a pair of BFS modules disposed between the barrel sections. For example, a first BFS module 210a (FIGS. 3A-3D) with a first fluid agent therein can be disposed within a first module recess 208a formed by the barrel sections and barrel partition 204, and a second BFS module 210b (FIGS. 4A-4D) with a second fluid agent therein can be disposed within a second module recess 208b formed by the barrel sections and barrel partition 204. In the illustrated example, the first BFS module 210a has a first BFS module neck portion 212a and a first module fluid reservoir 348. A first module fluid seal 342 is disposed at an axial end of the first BFS module neck portion 212a opposite from the first module fluid reservoir 348. Similarly, the second BFS module 210b has a second BFS module neck portion 212b and a second module fluid reservoir 448, and a second module fluid seal 442 is disposed at an axial end of the second BFS module neck portion 212b opposite from the second module fluid reservoir 448. The first BFS module 210a (e.g., first module fluid reservoir 348) can have a size and/or volume smaller than that of the second BFS module 210b (e.g., second module fluid reservoir 448). For example, a volume of the first fluid agent (e.g., atropine) in the first BFS module 210a can be about 0.7 mL, and a volume of the second fluid agent (e.g., pralidoxime chloride) in the second BFS module can be about 2 mL.

In some embodiments, the first BFS module 210a and/or the second BFS module 210b can include one or more features that engage with and/or mechanically couple to the barrel sections and/or the connector assembly 214, for example, to align the BFS modules with the respective piercing element and/or retain the BFS modules within the barrel sections and/or connector assembly 214. For example, first BFS module neck portion 212a can include a first module coupling feature 344 that is designed to engage with a first cooperative coupling feature 557a (e.g., recess, such as scalloped sidewall) of the connector assembly 214, and second BFS module neck portion 212b can include a second module coupling feature 444 that is designed to engage with a second cooperative coupling feature 557b (e.g., recess, such as a scalloped sidewall) of the connector assembly 214. Alternatively or additionally, in some embodiments, the BFS module can comprise and/or be coupled to one or more side ribs or protrusions, for example, first module flange 346 and/or second module flange 446 laterally extending from opposite side portions of the respective BFS module. The first and second module flanges may be designed to abut exterior facing surfaces of the first barrel section 202a and the second barrel section 202b (e.g., in an annular region disposed along the axial direction between the connector assembly 214 and the barrel sections), for example, as shown in FIG. 2D.

In some embodiments, either or both of the first BFS module 210a and/or the second BFS module 210b may be transparent in whole or in part to enable visualization of the fluid agent therewithin. In other embodiments, either or both of the first BFS module 210a and/or the second BFS module 210b may include transparent veins or windows. Visualization within the first BFS module 210a and/or the second BFS module 210b will enable the user to verify whether the fluid agent is fully or partially dispensed.

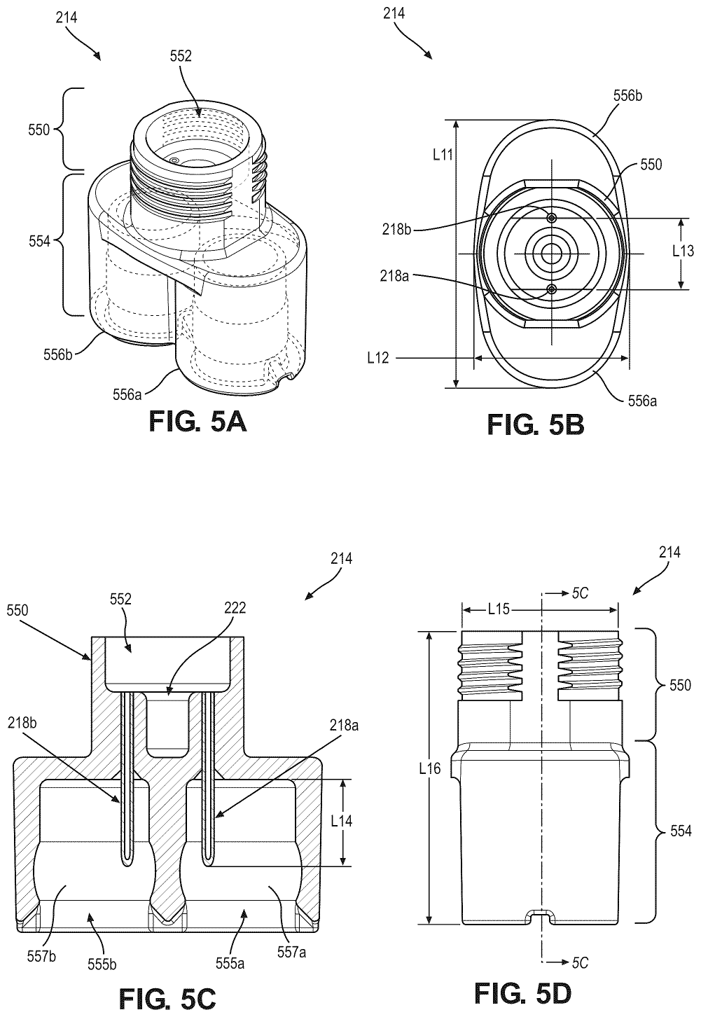

Referring to FIGS. 5A-5D, the connector assembly 214 can include a needle hub connector portion 550 and a module connector portion 554. The needle hub connector portion 550 can have a connection recess 552, to which the administration assembly 220 can be coupled in order to form conduit 222. In the illustrated example, an external surface of the needle hub connector portion 550 can be threaded, for example, to mate or engage with corresponding threads of the administration assembly 220. Alternatively or additionally, the internal surface of the needle hub connector portion 550 (e.g., within connection recess 552) can be threaded to mate or engage with corresponding threads of the administration assembly 220. In some embodiments, the module connector portion 554 includes one or more latches.

The module connector portion 554 can include a first module connector section 556a and a second module connector section 556b. The first module connector section 556a can define a first piercing region 555a, into which the first BFS module neck portion 212a of the first BFS module 210a can be inserted. An intermediate portion of a sidewall in the first piercing region 555a can serve as the first cooperative coupling feature 557a. Within the first piercing region 555a, a piercing end of the first piercing needle 218a can be disposed, while an opposite end (e.g., outlet end) of the first piercing needle 218a can be disposed within, or at least in fluid communication with, connection recess 552. Similarly, the second module connector section 556b can define a second piercing region 555b, into which the second BFS module neck portion 212b of the second BFS module 210b can be inserted, and an intermediate portion of a sidewall in the second piercing region 555b can serve as the second cooperative coupling feature 557b. A piercing end of the second piercing needle 218b can be disposed within the second piercing region 555b, and an opposite end (e.g., outlet end) of the second piercing needle 218b can be disposed within, or at least in fluid communication with, connection recess 552.

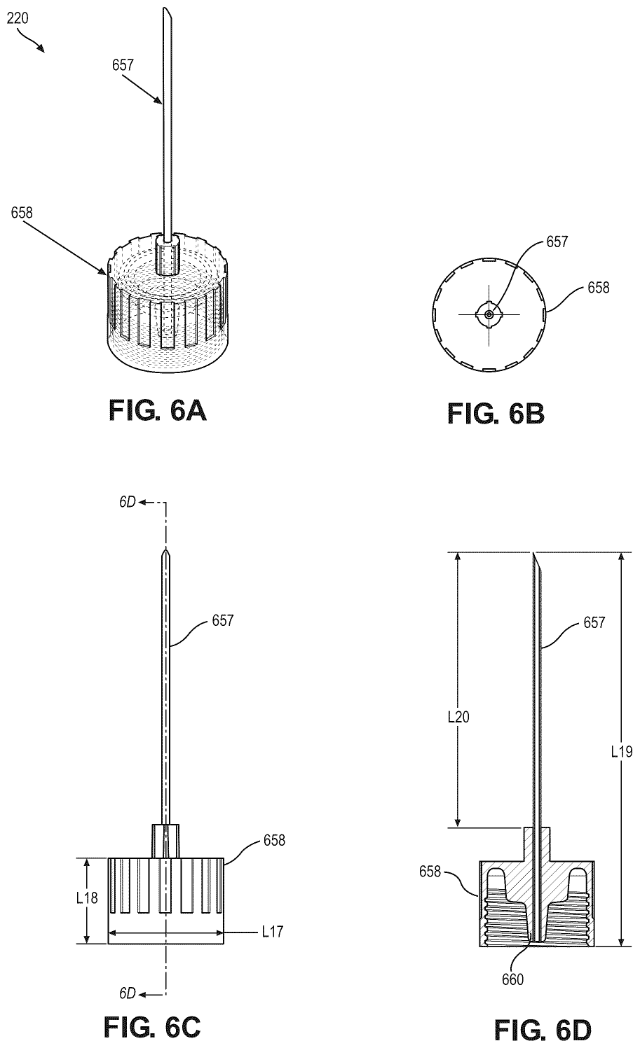

Referring to FIGS. 6A-6D, the administration assembly 220 can include a needle 657 and a needle hub 658. As shown in FIG. 6D, the needle hub 658 can have internal threads for engaging with external threads of the needle hub connector portion 550. Alternatively, in some embodiment, the needle hub 658 can have external threads for engaging with internal thread of the needle hub connector portion 550. In some embodiments, an inlet end of the needle 657 can be disposed closer to a back end (e.g., where plunger 224 is located) of the pre-filled dual-liquid medical delivery assembly 200 than the respective outlet ends of the first piercing needle 218a and second piercing needle 218b (e.g., an end of each needle opposite where fluid from the respective BFS module enters), for example, as shown in FIGS. 2E and 6D. In the illustrated example, the needle hub 658 includes an axially protruding member 660 that holds the inlet end of the needle 657 at the rearward disposition with respect to the piercing needle outlet ends, as well as defines (e.g., bounds) a portion of the conduit 222 (e.g., as shown in FIG. 2E).

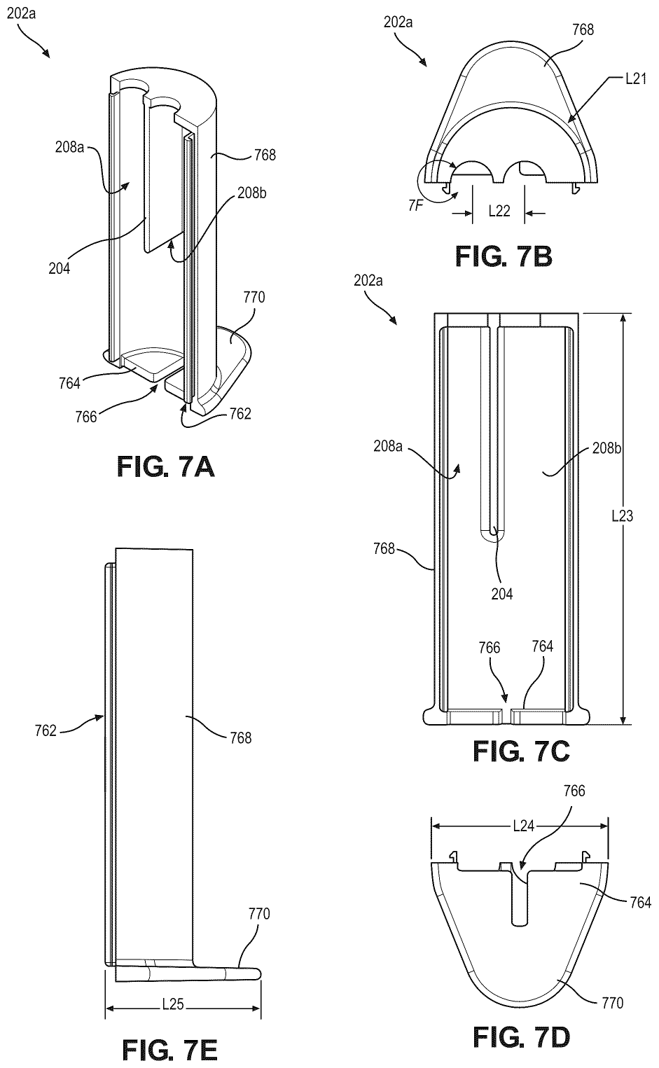

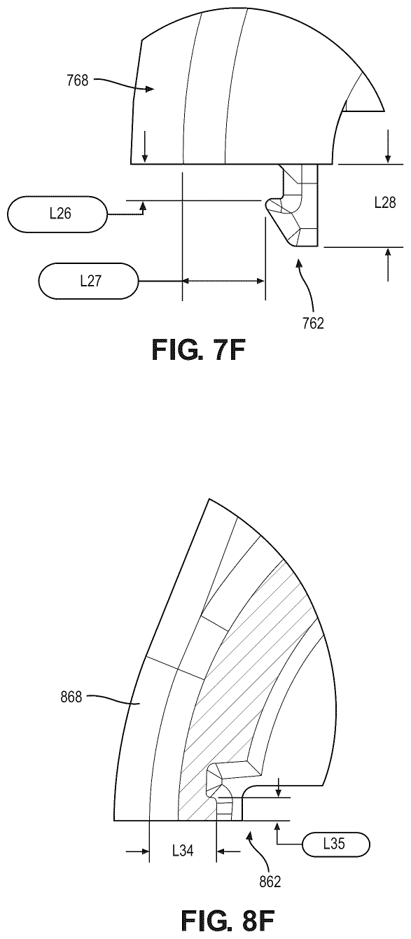

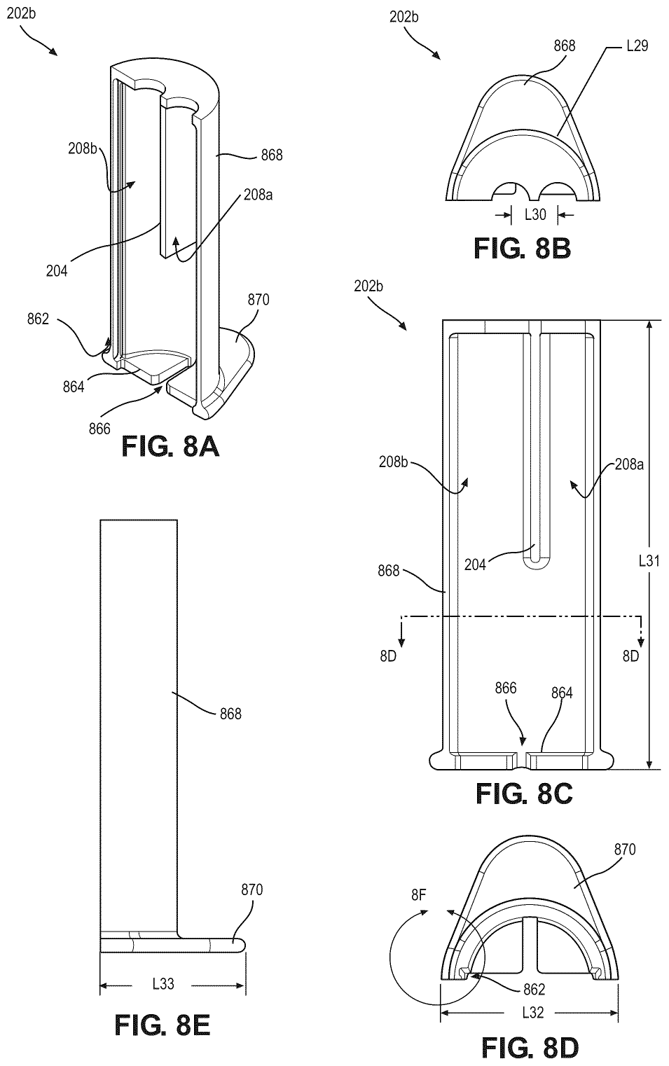

The first barrel section 202a can include a first barrel section recess cover portion 764 with a first barrel section plunger slot 766 therein, a first barrel section flange 770, and a first barrel section circumferential wall 768, as shown in FIGS. 7A-7F. Similarly, the second barrel section 202b can include a second barrel section recess cover portion 864 with a second barrel section plunger slot 866 therein, a second barrel section flange 870, and second barrel section circumferential wall 868, as shown in FIGS. 8A-8F. The first barrel section circumferential wall 768 can extend axially from the first barrel section recess cover portion 764 and the first barrel section flange 770 toward the connector assembly, and second barrel section circumferential wall 868 can extend axially from second barrel section recess cover portion 864 and second barrel section flange 870 toward the connector assembly. In some embodiments, the first barrel section flange 770 and second barrel section flange 870 can act finger holds for a user to apply force when depressing plunger 224. Portions of the plunger 224 can pass through first barrel section plunger slot 766 and second barrel section plunger slot 866 to allow axial motion of the plunger 224, while first barrel section recess cover portion 764 and second barrel section recess cover portion 864 can act to retain the plunger 224 within the barrel (e.g., prevent the plunger from being fully retracted out of the barrel) once fully assembled.

The first barrel section circumferential wall 768 and the second barrel section circumferential wall 868 can include one or more features designed to engage with each other, for example, to couple the first barrel section 202a and the second barrel section 202b together. For example, the first barrel section circumferential wall 768 can have a first barrel section connection feature 762 on axial edges thereof, and second barrel section circumferential wall 868 can have second barrel section connection feature 862 on axial edges thereof. For example, the first barrel section connection feature 762 can comprise a protrusion (e.g., locking tab or hook), as shown in FIG. 7F, and the second barrel section connection feature 862 can comprise a recess, as shown in FIG. 8F.

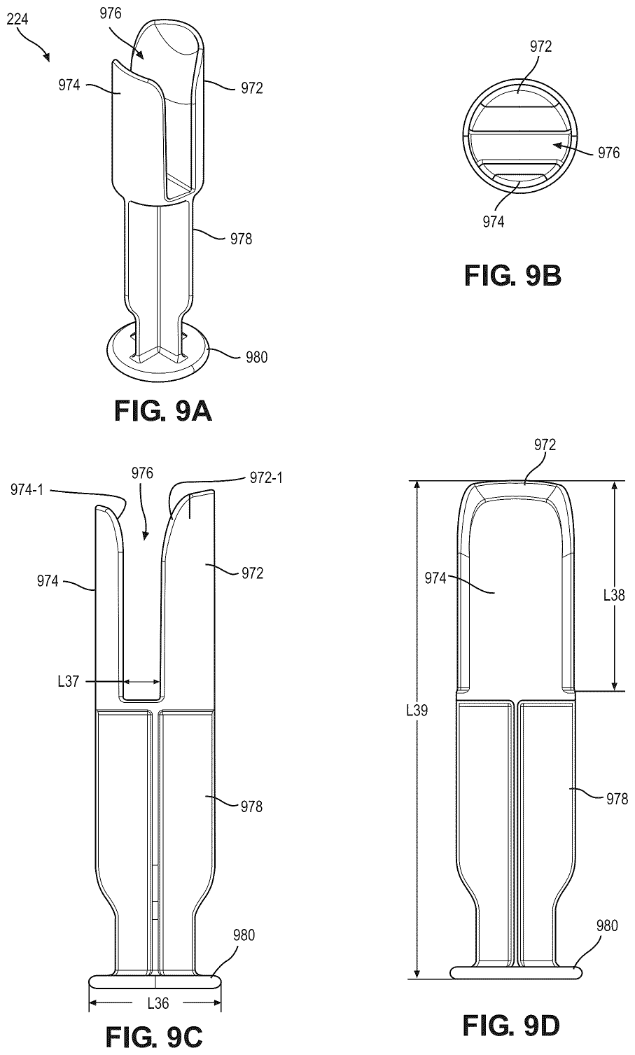

Referring to FIGS. 9A-9D, the plunger 224 can include a first module actuation portion 974, a second module actuation portion 972, an extension member 978, and an external actuation flange 980. The first module actuation portion 974 can be separated from the second module actuation portion 972 by a partition slot 976, for example, an axially-extending gap that corresponds to a location of barrel partition 204 within the barrel sections. The actuation portions can be disposed at an axial end of the extension member 978, and the external actuation flange 980 can be disposed at an opposite axial end of the extension member 978. The actuation portions and at least part of the extension member 978 can be disposed within the barrel sections, while the external actuation flange 980 can remain outside the barrel sections, for example, as shown in FIGS. 2B and 2D. The extension member 978 can be shaped so as to pass through the first barrel section plunger slot 766 and second barrel section plunger slot 866, for example, to allow axial movement of the plunger 224 toward the connector assembly 214.

In an exemplary operation, the first BFS module neck portion 212a of the first BFS module 210a can be inserted into the first piercing region 555a of the connector assembly 214, and the second BFS module neck portion 212b of the second BFS module 210b can be inserted into second piercing region 555b of the connector assembly 214. The exposed portions of the BFS modules (e.g., first module fluid reservoir 348 and second module fluid reservoir 448) can be disposed in the respective recesses of the first barrel section 202a (or second barrel section 202b), for example, with first module flanges 346 and/or second module flanges 446 abutting an upper surface of the respective barrel section. The actuation portions of the plunger 224 can also be disposed in the recesses of the first barrel section 202a (or second barrel section 202b) adjacent or at least proximal to the respective BFS modules. The second barrel section 202b (or first barrel section 202a) can then be placed over the first barrel section 202a and pushed together to engage the respective connection features. The needle hub 658 of administration assembly 220 can then be screwed on to the needle hub connector portion 550 of connector assembly 214, thereby forming conduit 222. The plunger 224 can then be pressed toward the connector assembly so as to inject fluid from the respective reservoirs into the conduit 222 and ultimately to the patient via needle 657.

In embodiments, the fluid from the reservoirs mix within at least the conduit 222 for delivery to the needle 657. In some embodiments, the fluid from each reservoir may mix in equal proportions (at a 1:1 ratio) for delivery to the patient. In other embodiments, the proportions may differ, for example, one reservoir may deliver more or less fluid agent to the conduit 222 than the other reservoir. In some embodiments, the fluid agent may mix at, for example, a 2:1 ratio.

In some embodiments, the first module actuation portion 974 and/or the second module actuation portion 972 can be tapered at an upper axial end (e.g., proximal to the connector assembly 214). For example, as shown in FIGS. 9B-9C, the first module actuation portion 974 can have a tapered end formed by a first sloped wall 974-1, and the second module actuation portion 972 can have a tapered end formed by a second sloped wall 972-1. In such a configuration, the sloped walls 972-1, 974-1 of the actuation portions 972, 974 can compress the fluid reservoirs of the BFS modules, for example, by pressing against a facing lateral surface of barrel partition 204. Alternatively or additionally, in some embodiments, axial displacement of the actuation portions 972, 974 toward the connector assembly 214 can press the BFS modules against an axial end surface of the barrel sections 202a, 202b (e.g., adjacent to connector assembly 214) and/or an axial end surface within the connector assembly 214.

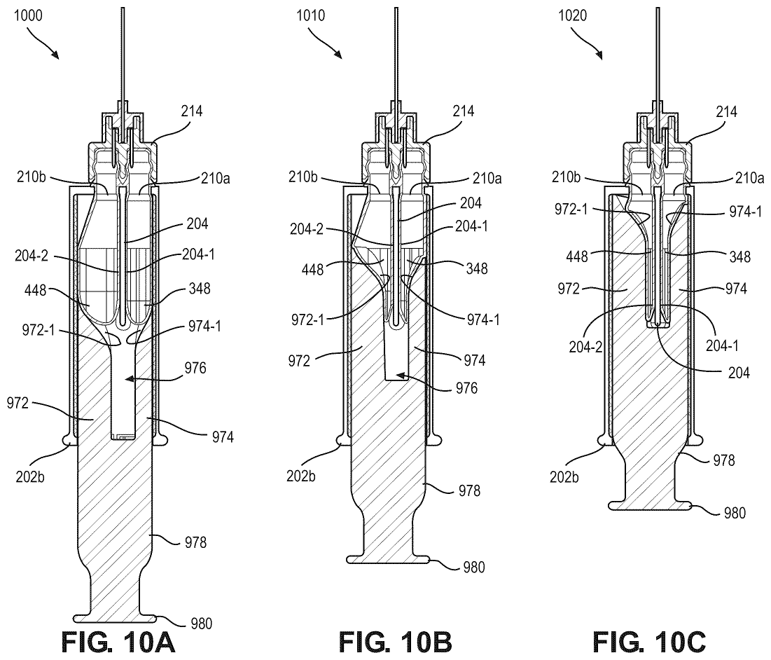

In some embodiments, in the initial state 1000 of FIG. 10A, the plunger 224 can be axially inserted into barrel sections 202a, 202b (only 202b shown in FIG. 10A) via an opening at end opposite connector assembly 214, such that first sloped wall 974-1 contacts first module fluid reservoir 348 and such that second sloped wall 972-1 contacts second module fluid reservoir 448. As shown by the partially-compressed state 1010 of FIG. 10B and the fully-compressed state 1020 of FIG. 10C, further axial displacement of the plunger toward the connector assembly 214 can compress the first module fluid reservoir 348 between the first sloped surface 974-1 and facing lateral surface 204-1 of barrel partition 204, and compresses the second module fluid reservoir 448 between the second sloped surface 972-1 and facing lateral surface 204-2 of barrel partition 204, thereby driving fluid from the first and second modules into the connector assembly 214. In some embodiments, the use of the sloped wall to compress the respective fluid reservoir against the barrel partition can ensure complete dispensing, or at least improve dispensing, of fluid from the fluid reservoir.

Although a particular order for assembly and use has been described above, embodiments of the disclosed subject matter are not limited thereto. Rather, different orders and/or variations in assembly or use are also possible according to one or more contemplated embodiments. For example, the administration assembly 220 can be coupled to the connector assembly 214 prior to connection with the BFS modules, after connection with the BFS modules but prior to securing the barrel sections, or at any other time. In another example, the BFS modules may be secured in the barrel sections prior to coupling to the connector assembly 214.

### III. Examples of Dimensions

Table 1 below presents exemplary dimensions for various components of a pre-filled medical delivery assembly according to one or more embodiments. However, other dimensions are also possible according to one or more contemplated embodiments. Accordingly, embodiments of the disclosed subject matter are not limited to the specific dimensions presented below.

TABLE 1
Exemplary Dimensions for Components in FIGS. 2A-10C

| Dimension | | Figure | Exemplary Value |
|---|---|---|---|
| Label | Component | Number | (mm) |
| L1 | Medical Delivery Assembly | FIG. 2B | 153.88 |
| L2 | Barrel Member Flange | FIG. 2B | 45.72 |
| L3 | First BFS Module | FIG. 3B | ∅ 7.30 |
| L4 | First BFS Module | FIG. 3C | 46.99 |
| L5 | First BFS Module | FIG. 3C | 12.70 |
| L6 | First BFS Module | FIG. 3D | 7.30 |
| L7 | Second BFS Module | FIG. 4B | ∅ 7.30 |
| L8 | Second BFS Module | FIG. 4C | 46.99 |
| L9 | Second BFS Module | FIG. 4C | 17.78 |
| L10 | Second BFS Module | FIG. 4D | 11.43 |
| L11 | Connector Assembly | FIG. 5B | 17.99 |
| L12 | Connector Assembly | FIG. 5B | 10.40 |
| L13 | Connector Assembly | FIG. 5B | 4.76 |
| L14 | Connector Assembly | FIG. 5C | 5.08 |
| L15 | Connector Assembly | FIG. 5D | ∅ 9.14 |
| L16 | Connector Assembly | FIG. 5D | 17.17 |
| L17 | Administration Assembly | FIG. 6C | ∅ 10.80 |
| L18 | Administration Assembly | FIG. 6C | 7.91 |
| L19 | Administration Assembly | FIG. 6D | 36.96 |
| L20 | Administration Assembly | FIG. 6D | 25.87 |
| L21 | First Barrel Member Section | FIG. 7B | ∅ 26.10 |
| L22 | First Barrel Member Section | FIG. 7B | 8.57 |
| L23 | First Barrel Member Section | FIG. 7C | 68.71 |
| L24 | First Barrel Member Section | FIG. 7D | 28.13 |
| L25 | First Barrel Member Section | FIG. 7E | 24.64 |
| L26 | First Barrel Member Section | FIG. 7F | 0.77 |
| L27 | First Barrel Member Section | FIG. 7F | 1.81 |
| L28 | First Barrel Member Section | FIG. 7F | 1.78 |
| L29 | Second Barrel Member Section | FIG. 8B | ∅ 26.10 |
| L30 | Second Barrel Member Section | FIG. 8B | 8.57 |
| L31 | Second Barrel Member Section | FIG. 8C | 68.71 |
| L32 | Second Barrel Member Section | FIG. 8D | 28.13 |
| L33 | Second Barrel Member Section | FIG. 8E | 22.86 |
| L34 | Second Barrel Member Section | FIG. 8F | 2.14 |
| L35 | Second Barrel Member Section | FIG. 8F | 0.74 |
| L36 | Plunger | FIG. 9C | 22.23 |
| L37 | Plunger | FIG. 9C | 6.10 |
| L38 | Plunger | FIG. 9D | 35.38 |
| L39 | Plunger | FIG. 9D | 83.89 |

### IV. Additional Embodiments

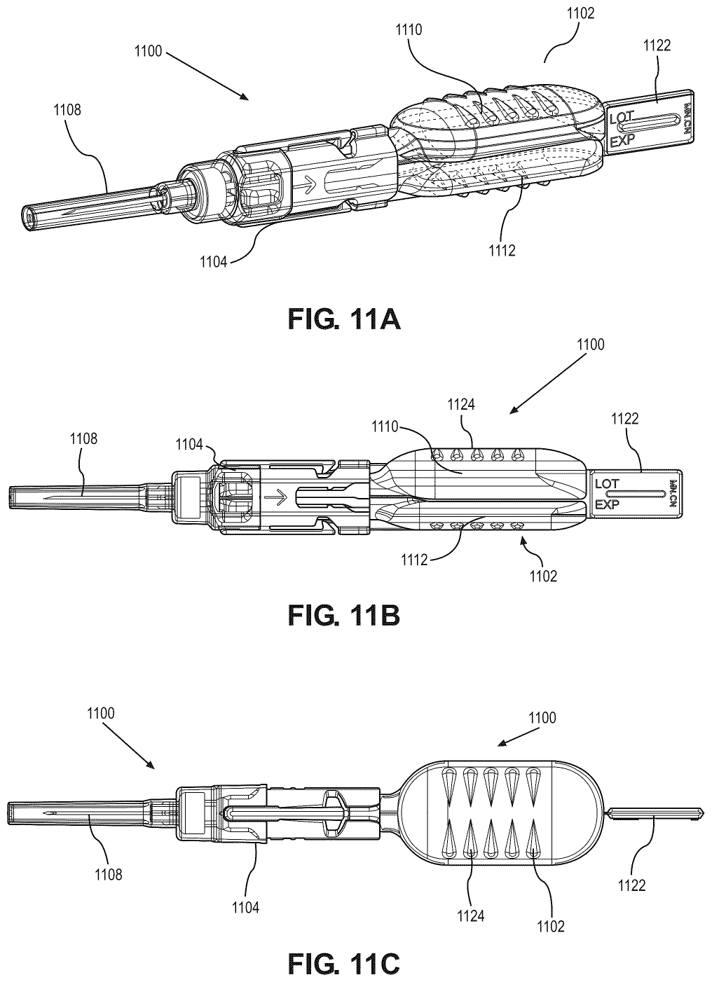

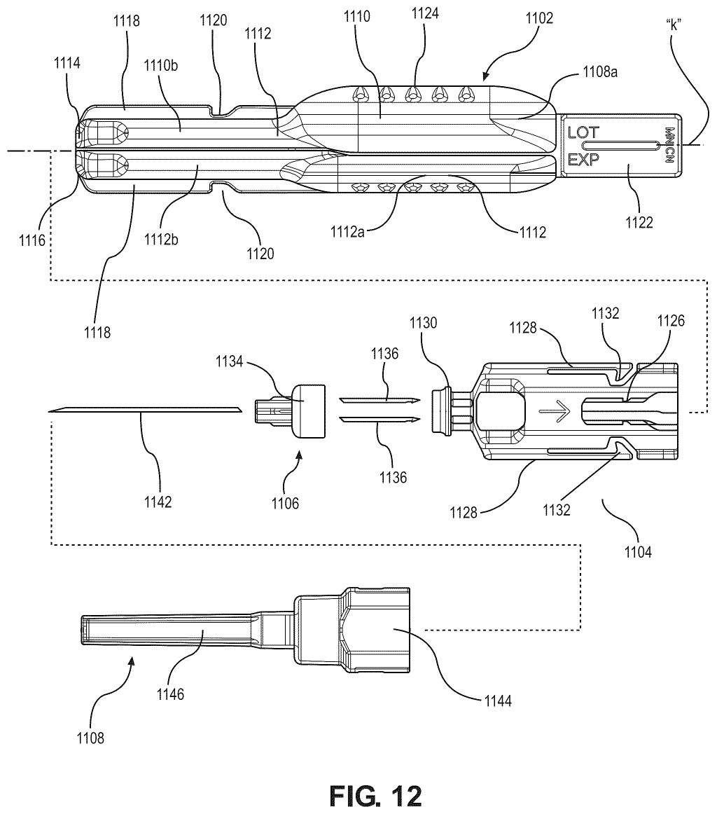

Referring now to FIGS. 11A-11C, in conjunction with FIG. 12, there is illustrated a pre-filled multi-fluid medical delivery assembly in accordance with one or more embodiments of the subject disclosure. The delivery assembly 1100 includes a dual chamber BFS vial 1102, a delivery module 1104 couplable to the BFS vial 1102, an administration hub assembly 1106 secured to the delivery module 1104 and a protective cover 1108 mountable over the delivery module 1104. The BFS vial 1102 includes a first vial module 1110 and a second vial module 1112 integrally formed with the first vial module 1110 and separated by a divider segment which extends along a majority of the length of the BFS vial 1102. The BFS vial 1102 including the first vial module 1110 and the second vial module 1112 may be constructed (e.g., formed) via a BFS process and may comprise a “soft” plastic (e.g., having a Shore/Durometer “OO” hardness of between 60 and 70 and/or a Shore/Durometer “A” hardness between 20 and 50) that is not functionally susceptible to the formation and/or utilization of threaded connection features (as are possible to form on different, harder plastics). During the BFS manufacturing process, the vial 1102 is formed, filled, and sealed in a continuous process without human intervention, in a sterile enclosed area inside a machine. In particular, the BFS manufacturing process involves multiple steps in which a pharmaceutical-grade plastic resin is vertically heat extruded through a circular opening to form a hanging tube called a parison. This extruded tube is then enclosed within a two-part mold, and the tube is cut above the mold. The mold is transferred to the filling zone, or sterile filling space, where filling needles (mandrels) are lowered and used to inflate the plastic to form the container within the mold. Following the formation of the vial 1102, the mandrels are used to fill the vial 1102 with one or more fluid agents. Following filling, the mandrels are retracted and a secondary top mold seals the container. All actions take place inside a sterile shrouded chamber inside the machine. The product is then discharged to a non-sterile area for labeling, packaging and distribution. In association with the BFS process, multiple vials 1102 are produced and connected to each other as one or more trays or units comprising rows and columns of vials 1102. Further details of compressible vials are illustrated in U.S. provisional patent Application Ser. No. 63/461,214, filed Apr. 21, 2023 and entitled “Syringe Cartridge System” and in international patent application publication number WO2017/187262 A1, published on 2 Nov. 2017 and entitled “Medical Delivery System,” the entire contents of each disclosure being incorporated by reference herein.

In embodiments, the first vial module 1110 and the second vial module 1112 are monolithically formed via the BFS process. More specifically, in embodiments, the first and second vial modules 1110, 1112 are not separate elements but are formed as a single unit comprised as part of the vial 1102. In other embodiments, the first and second vial modules 1110, 1112 may be separate components (for example, separate BFS components) connected to each other via conventional methodologies. The first vial module 1110 and the second vial module 1112 include respective first and second compressible main bodies 1110a, 1112a and first and second necks 1110b, 1112b extending longitudinally from the first and second main bodies 1110a, 1112b. In embodiments, the first and second compressible main bodies 1110a, 1112a collectively form the main body of the vial 1102 and the first and second necks 1110b, 1112b collectively form the neck of the vial 1102.

In embodiments, the first vial module 1110 defines a first fluid reservoir and the second vial module 1112 defines a second fluid reservoir independent from the first fluid reservoir. The first and second fluid reservoirs accommodate first and second fluid agents therein (not separately shown). According to some embodiments, the first and second fluid reservoirs of the first and second vial modules 1110, 1112 are filled with a different fluid agent (e.g., having different composition, phase, volume, or any combination of the foregoing), such as a different liquid or gas. Such different fluids agents can be combined, introduced, and/or mixed via the pre-filled multi-fluid medical system 1100 to define a combined fluid agent for use or administration to a patient. For example, the first vial module 1110 can contain a first fluid agent, and the second vial module 1112 can contain a second fluid agent. In some embodiments, the second fluid reservoir may house or contain a second volume (e.g., two to three milliliters (2.0 to 3.0-ml) of, for example, a vaccine or second agent (e.g., Pralidoxime). Alternatively or additionally, in some embodiments, the first fluid reservoir may house or contain a first volume (e.g., seven tenths milliliters (0.7-ml) of, for example, an adjuvant (and/or carrier fluid, catalyst, diluent, etc.) or first agent (e.g., Atropine).

According to some embodiments, the first fluid reservoir of the first vial module 1110 can have a first capacity (e.g., a volume for fluid agents), and/or the second vial module 1112 can have a second capacity (e.g., a volume for fluid agents). In some embodiments, the second capacity can be greater than the first capacity, and/or the volume of the second fluid agent contained within the second fluid reservoir can be greater than the volume of the first fluid agent contained within the first fluid reservoir. The second capacity can be up to twice or more greater than the first capacity. In some embodiments, the first and second vial modules 1110, 1112 may define the same capacity. According to some embodiments, any or all of the fluid agents may be injected into the first and second vial modules 1110, 1112 of the vial 1102 in a sterile environment during manufacture via a BFS process.

The first neck 1110b of the first vial module 1110 defines a first neck passage in fluid communication with the first fluid reservoir of the first vial module 1110 and terminates in a first penetrable seal 1114. The second neck 1112b of the second vial module 1112 defines a second neck passage in fluid communication with the second fluid reservoir of the second vial module 1112 and terminates in a second penetrable seal 1116. The first and second penetrable seals 1114, 1116 are longitudinally aligned relative to their respective first and second vial modules 1110, 1112, and thus are laterally spaced with respect to each other.

With reference to FIG. 12, in conjunction with FIGS. 11A-11C, the BFS vial 1102 may include a pair of diametrically opposed wings 1118 depending radially outwardly with respect to a longitudinal axis “k” of the vial 1102, and arranged in diametric opposed relation. The wings 1118 assist in aligning the vial 1102 with respect to the delivery module 1104 during coupling. The wings 1118 each include locking recesses 1120. The vial 1102 may further include a vial handle 1122. The vial handle 1122 may be planar and include indicia or an indicator (such as an RFID chip) representative of the type, lot, expiration date of the fluid agent contained within the BFS vial 1102. The wings 1118 and the vial handle 1122 may be formed during the BFS manufacturing process. The vial 1102 may comprise protrusions 1124 on the outer surfaces of the first and second vial modules 1110, 1112 to assist in handling of the vial 1102. The BFS vial 1102 may include transparent veins or windows to facilitate viewing within the reservoirs to check the volume of the fluid agents.

In some embodiments, the BFS vial 1102 may be specifically configured to achieve a squeeze force rating within a pre-defined squeeze force threshold. According to some embodiments, the BFS vial 1102 may be designed such that a squeeze-force of between thirty newtons (30-N) and forty-five newtons (45-N) is capable of compressing the first and second vial modules 1112, 1114 to such an extent that only a small percentage (e.g., less than twenty percent (20%), less than ten percent (10%), and/or less than five percent (5%)) and/or volume of fluid or medicament remains in the first and second vial modules 1112, 1114. Other squeeze forces for the BFS vial 1102 are contemplated.

The delivery module 1104 includes a connector main body 1126, a pair of locking arms 1128 mounted to the main body 1124 and a hub connector 1130 for coupling with the administration hub assembly 1106. The locking arms 1128 include locking detents 1132 which selectively engage the locking recesses 1120 of the BFS vial 1102 to secure the delivery module 1104 relative to the BFS vial 1102. In embodiments, the locking arms 1128 are normally biased to be received within the locking recesses 1120, but exhibit spring-like characteristics to flex outwardly during mounting of the delivery module 1104 on the vial 1102 and then return whereby the locking detents 1132 are received within the locking recesses 1120 thereby securing the delivery module 1104 on the BFS vial 1102.

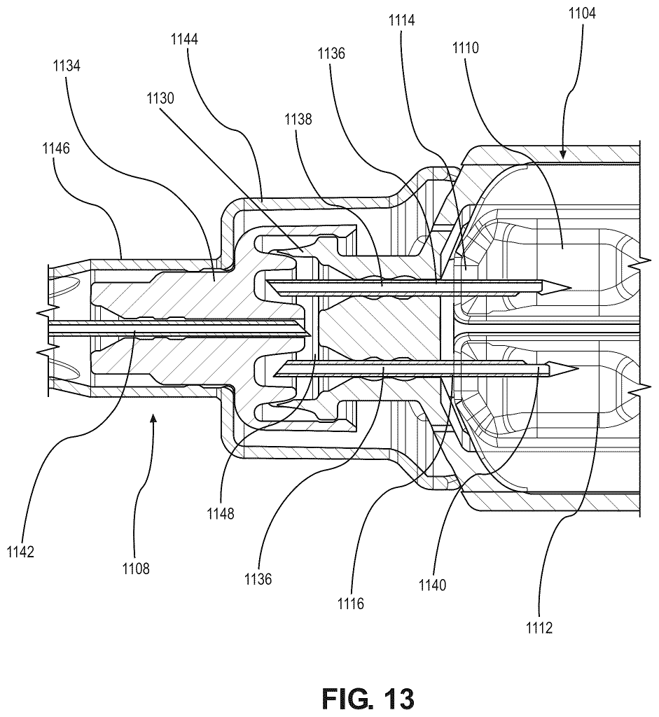

With reference to FIGS. 12 and 13, the administration hub assembly 1106 will be discussed. FIG. 13 is a partial cross-sectional view of the delivery module 1104, the administration hub assembly 1106 and the protective cover 1108. The administration hub assembly 1106 includes an administration hub 1134 which is configured to be secured to the hub connector 1130 of the delivery module 1104. In embodiments, the administration hub 1134 may be secured to the hub connector 1130 by conventional means including threading, a bayonet coupling, a snap fit, adhesives or the like. The administration hub 1134 includes first and second internal penetrating members 1136 depending within the delivery module 1104, and disposed in spaced relation. The first and second internal penetrating members 1136 may be needles, for example, hollow needles defining conduits, secured within the administration hub 1134 through conventional means including adhesives, cements, etc. In embodiments, the needles are received and secured within needle receiving openings 1138 in the administration hub 1134. The needles may include an axial opening or a side opening 1140 to access the internal reservoirs within the first and second vial modules 1110, 1112 to enable passage of the fluid agent therein and through the conduits of the needles. The first and second internal penetrating members 1136 are generally aligned with the first and second penetrable seals 1114, 1116 of the BFS vial 1102 to penetrate the first and second penetrable seals 1114, 1116 upon mounting/coupling of the delivery module 1104 on the BFS vial 1102. The administration hub 1134 further includes an external administration member 1142. The administration member 1142 may be a needle configured to administer the combined fluid agent from the first and second vial modules 1110, 1112 to a patient. In the alternative, the administration member 1142 may be a dropper or the like. The administration hub assembly 1106 may be preassembled or coupled to the delivery module 1104 prior to distribution to the operative site, for example, assembled at the manufacturing facility.

The protective cover 1108 includes a cover housing 1144 and an elongated cover component 1146 depending from the cover housing 1144. The cover housing 1144 includes inner wall portions defining an internal cover chamber configured to fit over the administration hub 1134 whereby, for example, an interference fit with the exterior of the administration hub 1134 is established between the components. The elongated cover component 1146 encloses the administration member 1142 during transport and prior to use. In some embodiments, the protective cover 1108 remains connected to the administration hub 1134 during coupling of the delivery module 1104 to the BFS vial 1102.

In use, the BFS vial 1102 and the delivery module 1104 with attached administration hub assembly 1106 and protective cover 1108 are distributed (independently or together) to the operative or treatment site. The delivery module 1104 is longitudinally aligned with the BFS vial 1102 such that the locking arms 1128 of the delivery module 1104 are aligned with the wings 1118 of the BFS vial 1102. In embodiments, the internal dimension of the connector main body 1126 and the wings 1118 of BFS vial 1102 are arranged whereby the BFS vial 1102 may only be introduced within the connector main body 1126 in the proper orientation with the wings 1118 aligned with the locking arms 1128 of the delivery module 1104. In other embodiments, the connector main body 1126 and/or the BFS vial 1102 may include indicia on its external surfaces facilitating arrangement in the proper orientation. The BFS vial 1102 is snapped into the connector main body 1126. During insertion of the BFS via 1102, the locking arms 1128 bias outwardly and then return (under their own resiliency) such that the locking detents 1132 of the locking arms 1128 are received within the locking recesses 1120 of the BFS vial 1102 in secured relation therewith. In conjunction therewith, the first and second internal penetrating members 1136 pierce, rupture or break the first and second penetrable seals 1114, 1116 enabling access to the fluid agent in each of the first and second vial modules 1110, 1112. The protective cover 1108 may be removed.

With the first and second internal penetrating members 1136 accessing the first and second vial modules 1110, 1112 as shown in FIG. 13, a squeezing action is exerted on the first and second vial modules 1110, 1112 to cause the fluid agents from each of the first and second vial modules 1110, 1112 to mix within the internal mixing chamber 1148 defined between the within the administration hub 1134 of the administration hub assembly 1106 and the hub connector 1130 for mixing and distribution through the administration member 1142. In embodiments, the fluid agents flow through the conduits of the first and second internal penetrating members 1136 to mix within the internal mixing chamber 1148 prior to passage through the administration member 1142.

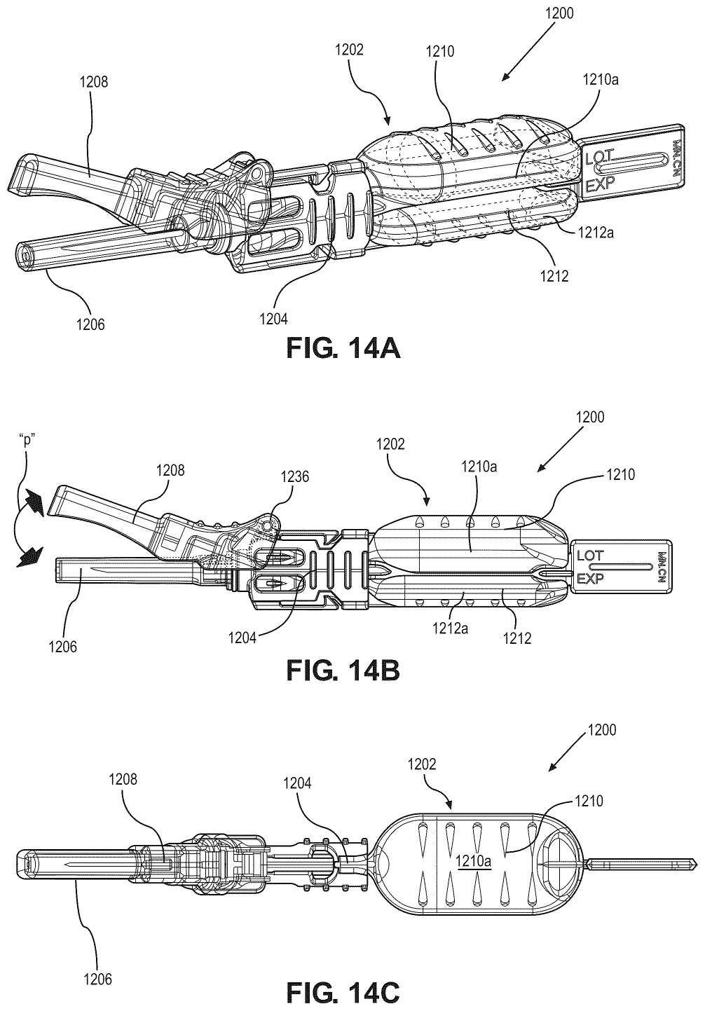

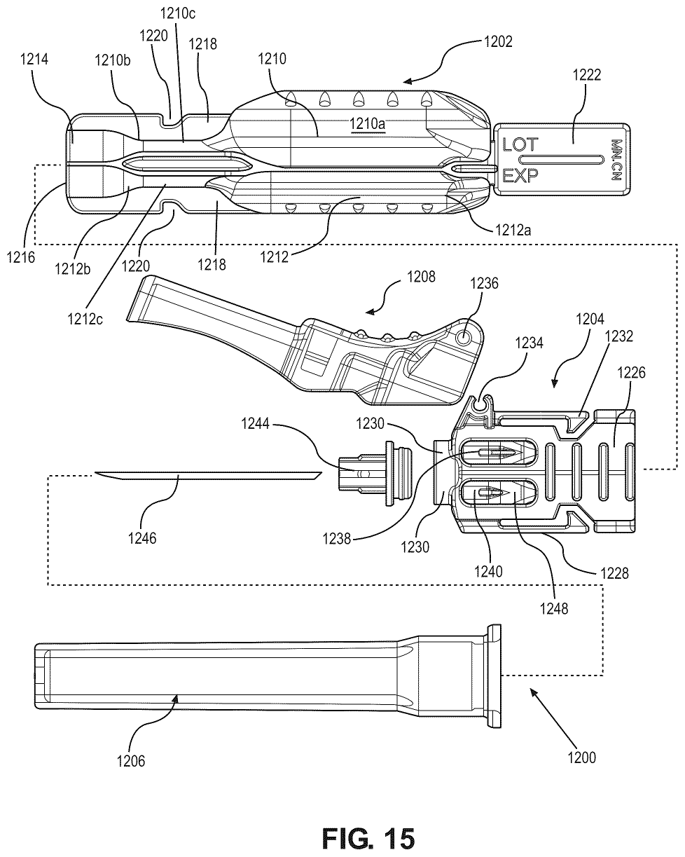

Referring now to FIGS. 14A-14C, in conjunction with FIG. 15, there is another illustrated pre-filled multi-fluid medical delivery assembly in accordance with one or more embodiments of the present disclosure. The delivery assembly 1200 is similar in at least some respects to the delivery assembly 1100 of FIGS. 11A-13, and reference is made to the previous description for a description of some of the similar components. The delivery assembly 1200 includes a BFS vial 1202, a delivery module 1204 coupled to the BFS vial 1202, a protective cover 1206 coupled to the delivery module 1204 and a pivoting shield 1208 also coupled to the delivery module 1204.

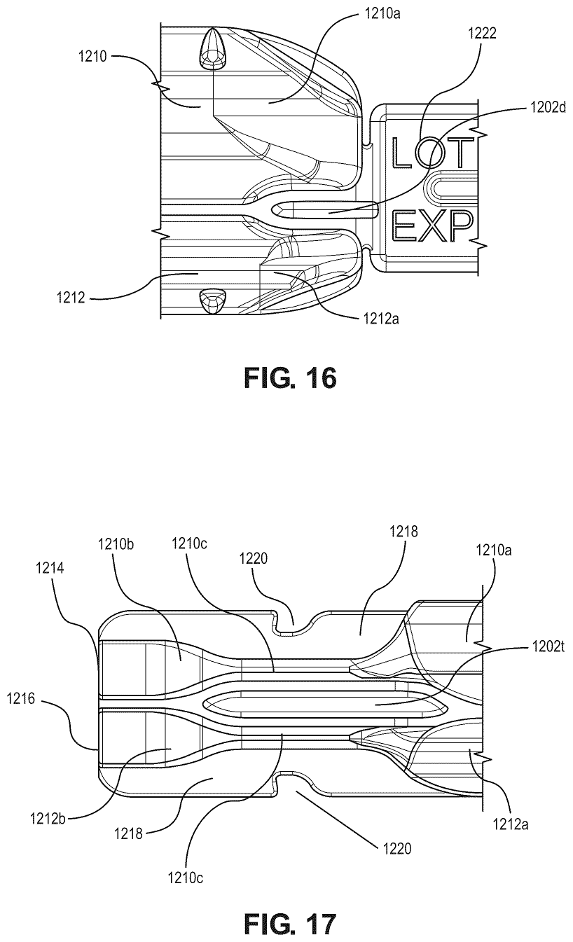

As best depicted in FIGS. 15-17, the BFS vial 1202 includes a first vial module 1210 and a second vial module 1212 integrally formed with the first vial module 1210 and separated by a divider segment which extends along a majority of the length of the BFS vial 1102. The first vial module 1210 and the second vial module 1212 include respective first and second compressible main bodies 1210a, 1212a and first and second necks 1210b, 1212b extending longitudinally from the first and second main bodies 1210a, 1212b. In embodiments, the first and second compressible main bodies 1210a, 1212a collectively form the main body of the vial 1202 and the first and second necks 1210b, 1212b collectively form the neck of the vial 1202. The first and second necks 1210b. 1212b define a fluted section at the remote end having an internal dimension or conduit greater than the conduit 1210c, 1212c extending from the main bodies 1210a, 1212a. The first and second necks 1210b. 1212b terminate in penetrable seals 1214, 1216 respectively. The BFS vial 1202 may include an enlarged divider segment 1202d between the first and second vial modules 1210, 1212. (FIG. 16). The enlarged divider segment 1202d may assist in maintaining the integrity of the BFS vial 1202 while removing the BFS vial 1202 from a strip of BFS vials and/or during manipulating of the BFS vial 1202 relative to the delivery module 1204. In some embodiments, the divider segment 1202d may resist any tendency of tearing and/or leaking of the BFS vial 1202 during use. The enlarged divider segment 1202d may be formed during the BFS molding process. The BFS vial 1202 may further include a depressed or thinned segment 1202t between the conduits 1210c, 1212c of the first and second vial modules 1210, 1212. The thinned segment 1202t may be formed during the BFS molding process, and may be a result of material transfer of the BFS plastic material along at least the conduits 1210c, 1212c. This maintains separation between the chambers. The BFS vial 1202 may include a pair of diametrically opposed wings 1218 depending radially outwardly with respect to axis of the vial 1202, and arranged in diametric opposed relation. The wings 1218 each include locking recesses 1220. The vial 1202 may further include a vial handle 1222 and may include protrusions 1224 on the outer surfaces of the first and second vial modules 1210, 1212 to assist in handling of the vial 1102.

With reference again to FIG. 15, the delivery module 1204 includes a connector main body 1226, a pair of locking arms 1228 mounted to the main body 1224 and an administration hub connector 1230. The locking arms 1228 include locking detents 1232 which selectively engage the locking recesses 1220 of the BFS vial 1202 to secure the delivery module 1204 relative to the BFS vial 1202. The delivery module 1204 further includes a recessed mount 1234 for securing the pivoting shield 1208 to enable pivotal movement of the pivoting shield 1208. In embodiments, the recessed mount 1234 is dimensioned to receive a corresponding dimensioned pivot pin 1236 of the protective shield 1208 to mount the pivoting shield 1208 in pivotal relation to the delivery module 1204. The pivoting shield 1208 may reciprocally pivot in the direction “p” shown in FIG. 14B.

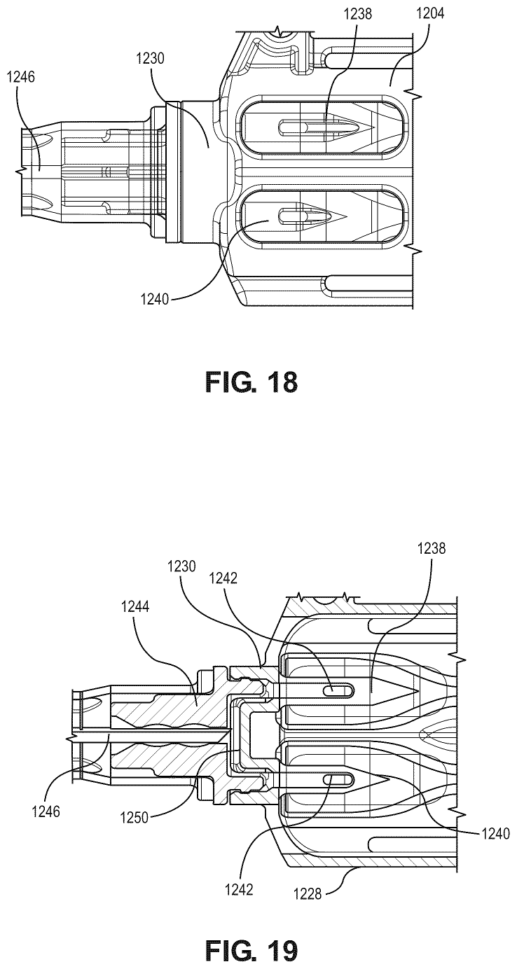

As best depicted in FIGS. 15, 18 and 19, the delivery module 1204 further includes first and second internal penetrating members 1238, 1240 extending within the interior of the delivery module 1204. FIGS. 18 and 19 are partial side elevation and cross-sectional views respectively of the delivery module 1204 and the BFS vial 1202. The first and second penetrating members 1238, 1240 may be integrally or monolithically formed with the delivery module 1204. In embodiments, the delivery module 1204 and the first and second internal penetrating members 1238, 1240 are monolithically formed of a suitable polymeric material via conventional molding or techniques. The first and second internal penetrating members 1238, 1240 may be staggered, e.g., the first penetrating member 1238 may be longer than the second penetrating member 1240. With this arrangement, the first penetrating member 1238 will penetrate the first penetrable seal 1214 of the first vial module 1210 followed by penetration of the second penetrable seal 1216 of the second vial module 1212 with the second internal penetrating member 1240, i.e., in succession. This effectively reduces installation/penetration forces required to couple and actuate the components. The first and second internal penetrating members 1238, 1240 may include side and/or axial ports 1242 for introduction and passage of the fluid agent within each of the respective first and second vial modules 1210, 1212.

The delivery module 1204 further includes an administration hub 1244 for coupling with the administration hub connector 1230. The administration hub 1244 may be coupled to the hub connector 1230 by any suitable coupling methodologies including a threaded coupling, bayonet coupling, snap fit, adhesives or the like. An administration member 1246 is secured to the administration hub 1244. The administration member 1246 may be in the form of a needle or a dropper. The delivery module 1204 may further include one or more windows 1248 to enable viewing of the BFS vial 1202 to ensure proper coupling with the delivery module 1204, proper penetration of the first and second internal penetrating members 1238, 1240 through the first and second penetrable seals 1214, 1216 or to view the volume of the fluid agent within the first and second vial modules 1210, 1212.

In use, the BFS vial 1202 and the delivery module 1204 with attached protective cover 1206 and protective shield 1208 are distributed to the operative or treatment site. The delivery module 1204 is longitudinally aligned with the BFS vial 1202 such that the locking arms 1228 of the delivery module 1204 are aligned with the wings 1218 of the BFS vial 1102. The BFS vial 1202 is snapped into the connector main body 1226 of the delivery module 1204. During insertion of the BFS vial 1202, the locking arms 1228 bias outwardly and then return (under their own resiliency) such that the locking detents 1232 of the locking arms 1228 are received within the locking recesses 1220 of the BFS vial 1202 in secured relation therewith. In conjunction therewith, the first and second internal penetrating members 1238, 1240, rupture or break the first and second penetrable seals 1214, 1216, in, for example, sequence, enabling access to the fluid agent in each of the first and second vial modules 1210, 1212. As mentioned above, staggering of the first and second internal penetrating members 1238, 1240 will reduce penetration and assembly forces. With the first and second internal penetrating members 1136 accessing the first and second vial modules 1110, 1112, the protective cover 1206 may be removed. A squeezing action is exerted on the first and second vial modules 1210, 1212 to cause the fluid agent from each of the first and second vial modules 1210, 1212 to mix within the internal mixing chamber 1250 defined between the administration hub 1244 and the administration hub connector 1130 for distribution through the administration member 1246. (FIG. 19) Any time during use, the protective shield 1208 may be selectively pivoted in the direction of directional arrow “p” (FIG. 14B) to cover the administration member 1246 and/or to selectively expose the administration member 1246.

In some embodiments, the pivoting shield 1208 may include an internal locking latch (not shown) which may be configured to selectively secure the administration member 1246 when the pivoting shield 1208 is pivoted to cover the administration member 1246. In some embodiments, the locking latch may prevent reuse of the administration member 1246, i.e., the pivoting shield cannot be pivoted off the administration member 1246 once secured to the administration member 1246 without deforming and/or breaking the locking latch and/or the administration member 1246. In other embodiments, the locking latch permits movement of the pivoting shield 1208 off the administration member 1246 subsequent to engagement with the administration member 1246.

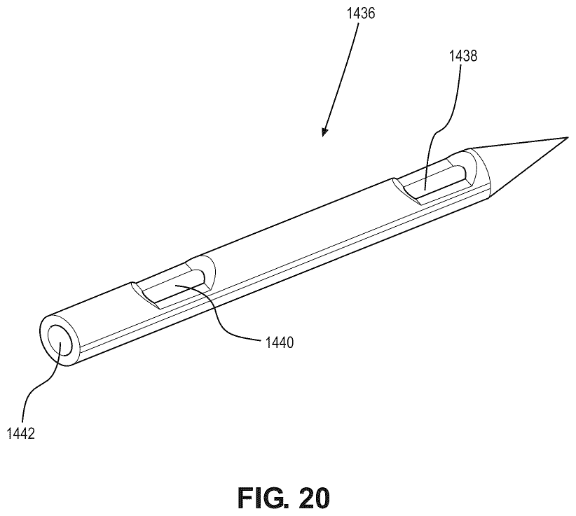

FIG. 20 is a partial perspective view of an internal penetrating member 1436 for use with the delivery modules 1104, 1204 for penetrating the first and second vial modules 1110, 1112, 1210, 1212. The internal penetrating member 1436 may include first and second side ports 1438, 1440 and internal conduit 1442 in fluid communication with the first and second side ports 1438, 1440. The internal conduit 1442 deposits into the internal mixing chambers prior to distribution through the administration member. The internal penetrating member 1436 is dimensioned to pierce the first and second penetrable seals 1114, 1116, 1214, 1216. The presence of the first and second side ports 1438, 1440 may, in embodiments, increase flow of the fluid agent through the internal penetrating member 1436.

### V. Rules of Interpretation

Any or all of the components of the medical delivery assembly disclosed herein can be formed of one or more plastics. In some embodiments, some components (e.g., the BFS modules) can be formed of a relatively soft polymer (e.g., having a Shore/Durometer “D” hardness of between 60 and 70), such as polyethylene (e.g., low density polyethylene (LDPE)), polypropylene, or any other polymer adaptable for use in a BFS manufacturing process. In some embodiments, some components (e.g., the syringe barrel member and/or sections, the barrel partition, the delivery module assembly, the patient needle assembly, and/or the plunger) can be formed, at least in part, of a relatively hard polymer (e.g., having a hardness greater than 80 on the Rockwell “R” scale), such as, but not limited to, polypropylene, polycarbonate, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments. A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required. Although a product may be described as including a plurality of components, aspects, qualities, characteristics and/or features, that does not indicate that all of the plurality are essential or required. Various other embodiments within the scope of the described invention(s) include other products that omit some or all of the described plurality. A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Neither the Title (set forth at the beginning of the first page of this patent application) nor the Abstract (set forth at the end of this patent application) is to be taken as limiting in any way as the scope of the disclosed invention(s). Headings of sections provided in this patent application are for convenience only, and are not to be taken as limiting the disclosure in any way.

All definitions, as defined and used herein, should be understood to control over dictionary definitions, definitions in documents incorporated by reference, and/or ordinary meanings of the defined terms. The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

While the term “modules” is utilized herein for convenience and ease of illustration, objects represented and/or described as “modules” may comprise various forms, configurations, and/or quantities of components. A BFS module may comprise one or more BFS products that are formed and/or manufactured together or separately, for example, and/or may comprise one or more BFS vials, cartridges, chambers, bottles, containers, and/or other fluid-retaining objects. The term “module” does not convey any designation of shape or size. In some embodiments, a BFS module may comprise one or more vials. According to some embodiments, a BFS module may comprise one or more fluid chambers. In some embodiments, a plurality of BFS modules, components, and/or chambers may be manufactured simultaneously from a single BFS mold. Each respective module, component, and/or chamber may be formed, for example, by different portions of a single BFS mold (e.g., two cooperative halves thereof). In some embodiments, BFS modules, components, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise. Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

A reference to “another embodiment” in describing an embodiment does not imply that the referenced embodiment is mutually exclusive with another embodiment (e.g., an embodiment described before the referenced embodiment), unless expressly specified otherwise.

The indefinite articles “a” and “an,” as used herein in the specification and in the claims, unless clearly indicated to the contrary, should be understood to mean “at least one” or “one or more”.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car, and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. Similarly, while the terms “horizontal” and “vertical” may be utilized herein, such terms may refer to any normal geometric planes regardless of their orientation with respect to true horizontal or vertical directions (e.g., with respect to the vector of gravitational acceleration).

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

### VI. Conclusion

Any of the features illustrated or described with respect to FIGS. 1-20 can be combined with any other features illustrated or described with respect to FIGS. 1-20 to provide systems, assemblies, kits, devices, methods, and embodiments not otherwise illustrated or specifically described herein. All features described herein are independent of one another and, except where structurally impossible, can be used in combination with any other feature described herein.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly comprising:
(i) a first blow-fill-seal (BFS) module having a first fluid seal and a first reservoir with a first fluid agent therein;
(ii) a second BFS module having a second fluid seal and a second reservoir with a second fluid agent therein;
(iii) a barrel member defining first and second recesses for receiving therein the first and second BFS modules, respectively;
(iv) a connector assembly having first and second piercing regions, each piercing region having a piercing member therein, the first piercing region being disposed with respect to the first recess of the barrel member, the second piercing region being disposed with respect to the second recess of the barrel member;
(v) an administration assembly comprising an administration conduit, the administration assembly being coupled to the connector assembly such that the administration conduit is in fluid communication with each of the first and second piercing regions; and
(vi) a plunger configured to move axially with respect to the barrel member, the plunger having first and second actuation portions disposed within the barrel member and constructed to engage with the first and second BFS modules, respectively,
wherein the pre-filled medical delivery assembly is configured such that axial movement of the plunger relative to the administration assembly causes the piercing members of the first and second piercing regions to penetrate the first and second fluid seals, respectively to permit distribution of the first fluid agent and the second fluid agent through the administration conduit.

2. The pre-filled medical delivery assembly of claim 1, wherein a volume of the first reservoir is different than a volume of the second reservoir, and/or a volume of the first fluid agent is different than a volume of the second fluid agent.

3. The pre-filled medical delivery assembly of claim 2, wherein a volume of the second reservoir is at least 2 times greater than a volume of the first reservoir, and/or a volume of the second fluid agent is at least 2 times greater than a volume of the first fluid agent.

4. The pre-filled medical delivery assembly of claim 3, wherein a volume of the second fluid agent is about 2 ml, and a volume of the first fluid agent is about 0.7 ml.

5. The pre-filled medical delivery assembly of claim 1, wherein at least one of the first fluid agent or the second fluid agent comprises one of a vaccine, a drug, a medicament, or a component of any of the foregoing.

6. The pre-filled medical delivery assembly of claim 1, wherein at least one of the first fluid seal or the second fluid seal comprises a foil, wax, paper, a reduced-thickness section of the respective BFS module, or any combination of the foregoing.

7. The pre-filled medical delivery assembly of claim 1, wherein the administration conduit comprises a needle or cannula.

8. The pre-filled medical delivery assembly of claim 7, wherein the needle has a length in a range of 0.5 mm to 4 mm, inclusive, or in a range of 4 mm to 15 mm, inclusive, or in a range of 15 mm to 30 mm, inclusive.

9. The pre-filled medical delivery assembly of claim 1, wherein an outlet of the administration conduit is formed as a nozzle configured to facilitate dispersion of a combination of the first and second fluid agents into a spray or one or more droplets.

10. The pre-filled medical delivery assembly of claim 1, wherein one, some, or all of the barrel member, the connector assembly, and the plunger are formed of a respective material having a hardness greater than that of the first BFS module, the second BFS module, or both the first and second BFS modules.

11. The pre-filled medical delivery assembly of claim 1, wherein the connector assembly and the administration assembly are coupled together so as to define an internal conduit between the connector assembly and the administration assembly, and the internal conduit fluidically connects the first and second reservoirs to the administration conduit via the respective first and second piercing regions.

12. The pre-filled medical delivery assembly of claim 11, wherein:
the internal conduit has a first section adjacent to an outlet from the first piercing region, a second section adjacent to an outlet from the second piercing region, and a third section adjacent to an inlet of the administration conduit; and
the third section is disposed between the first and second sections.

13. The pre-filled medical delivery assembly of claim 12, wherein the third section is disposed at a midpoint between the first and second sections.

14. The pre-filled medical delivery assembly of claim 13, wherein the third section is disposed between the first and second sections with respect to a radial direction of the administration conduit.

15. The pre-filled medical delivery assembly of claim 14, wherein the third section is disposed between the first section and the first piercing region and/or between the second section and the second piercing region with respect to an axial direction of the administration conduit.

16. The pre-filled medical delivery assembly of claim 1, wherein each piercing member comprises a needle.

17. The pre-filled medical delivery assembly of claim 1, wherein the barrel member comprises an axially-extending internal partition that divides the first recess from the second recess.

18. The pre-filled medical delivery assembly of claim 17, wherein at least part of the first actuation portion is tapered so as to compress the first reservoir against the internal partition as the plunger moves axially toward the administration assembly, and/or at least part of the second actuation portion is tapered so as to compress the second reservoir against the internal partition as the plunger moves axially toward the administration assembly.

19. The pre-filled medical delivery assembly of claim 1, wherein the barrel member comprises a pair of lateral sections coupled together.

20. The pre-filled medical delivery assembly of claim 1, wherein the first BFS module comprises a first neck portion with the first seal at an end thereof, and the second BFS module comprises a second neck portion with the second seal at an end thereof.

21. The pre-filled medical delivery assembly of claim 20, wherein:
the first neck portion comprises a first mating feature, and the first piercing region comprises a first cooperating feature configured to engage with the first mating feature; and
the second neck portion comprises a second mating feature, and the second piercing region comprises a second cooperating feature configured to engage with the second mating feature.

22. The pre-filled medical delivery assembly of claim 21, wherein:
the first mating feature comprises a first laterally-protruding portion; and
the second mating feature comprises a second laterally-protruding portion.

23. The pre-filled medical delivery assembly of claim 22, wherein:
the first mating feature has a toroidal shape in a side view, and the first cooperating feature comprises a scalloped portion of a sidewall of the first piercing region; and
the second mating feature has a toroidal shape in a side view, and the second cooperating feature comprises a scalloped portion of a sidewall of the second piercing region.

24. The pre-filled medical delivery assembly of claim 1, wherein the first BFS module and the second BFS module each comprises one or more flanges constructed to engage with one of the barrel member or the connector assembly.

25. The pre-filled medical delivery assembly of claim 1, wherein:
a first longitudinal direction of the first BFS module extends from the first reservoir toward the first fluid seal;
a second longitudinal direction of the second BFS module extends from the second reservoir toward the second fluid seal; and
the first and second longitudinal directions are substantially parallel to and spaced apart from each other along a radial direction.

26. The pre-filled medical delivery assembly of claim 25, wherein the first and second longitudinal directions are on opposite sides of the administration conduit.

27. The pre-filled medical delivery assembly of claim 1, wherein at least part of the piercing members of the first and second piercing regions overlap with at least part of the administration conduit.

28. The pre-filled medical delivery assembly of claim 1, wherein the first piercing region and the second piercing region are staggered wherein a first piercing end of the first piercing region is longitudinally displaced relative to a second piercing end of the second piercing region.

29. A pre-filled medical delivery assembly comprising:
(i) a first blow-fill-seal (BFS) module having a first fluid seal and a first reservoir with a first fluid agent therein;
(ii) a second BFS module having a second fluid seal and a second reservoir with a second fluid agent therein;
(iii) a barrel member for receiving therein the first and second BFS modules, and defining a longitudinal axis;
(iv) a connector assembly having first and second piercing regions, the first piercing region having a first piercing end, the second piercing region having a second piercing end displaced longitudinally with respect to the first piercing end of the first piercing region;
(v) an administration assembly comprising an administration conduit, the administration assembly being coupled to the connector assembly such that the administration conduit is in fluid communication with each of the first and second piercing regions; and
(vi) a plunger constructed to move axially with respect to the barrel member, to engage the first and second BFS modules to cause the piercing members of the first and second piercing regions to penetrate, in succession, the first and second fluid seals, respectively.

30. The pre-filled medical delivery assembly of claim 29, wherein the barrel member defines first and second recesses for receiving therein the first and second BFS modules, respectively.

31. A method, comprising:
introducing a blow fill seal component into a barrel component, the blow fill seal component including a first blow-fill-seal (BFS) module having a first fluid seal and a first reservoir with a first fluid agent therein and a second BFS module having a second fluid seal and a second reservoir with a second fluid agent therein;
at least partially positioning a plunger component into the barrel component; and
advancing the plunger component within the barrel component along an axis of the barrel component to cause a first internal penetration member associated with the barrel component to penetrate the first fluid seal of the first BFS module and a second internal penetration member associated with the barrel component to penetrate the second fluid seal of the second BFS module, to cause release of the first fluid agent and the second fluid agent for distribution through an administration component coupled relative to the barrel component.

32. The method of claim 31, including coupling the administration component relative to the barrel component.

33. The method of claim 32, including mixing the first fluid agent and the second fluid agent within a conduit in fluid communication with the administration component.

34. The method of claim 33, wherein introducing a blow seal component includes at least partially positioning the first BFS module into a first barrel recess of the barrel component and the second BFS module into a second barrel recess of the barrel component.

35. The method of claim 34, including coupling a connector component to the barrel component, the connector component including the first and second internal penetration members and the conduit.

36. The method of claim 35, wherein the first internal penetration member is axially displaced relative to the second internal penetration member, wherein advancing the plunger component includes the first internal penetration member penetrating the first fluid seal prior to the second internal penetration member penetrating the second fluid seal.

37. A pre-filled medical delivery assembly comprising:
a blow-fill-seal (BFS) vial including a first vial module having a first fluid seal and a first reservoir with a first fluid agent therein and a second vial module having a second fluid seal and a second reservoir with a second fluid agent therein, the BFS vial defining a longitudinal axis;
a delivery module couplable to the BFS vial including:
a main body for at least partially receiving the BFS vial;
a first internal penetrating member disposed within the main body and defining a first fluid conduit; and
a second internal penetrating member disposed within the main body and defining a second fluid conduit; and
an administration member mounted to the delivery module;
wherein upon coupling of the BFS vial and the delivery module:
the first internal penetrating member penetrates the first fluid seal of the first vial module and the second internal penetrating member penetrates the second fluid seal of the second vial module; and
the administration member is in fluid communication with the first fluid conduit of the first internal penetrating member and in fluid communication with the second fluid conduit of the second internal penetrating member to enable delivery of the first and second fluid agents through the administration member.

38. The pre-filled delivery assembly of claim 37 wherein the first internal penetrating member is axially aligned with the first fluid seal of the first vial module and the second internal penetrating member is axially aligned with the second fluid seal of the second vial module upon coupling of the BFS vial and the delivery module.

39. The pre-filled delivery assembly of claim 37 wherein the main body includes a pair of locking arms with locking detents, the locking detents being received within cooperating locking recesses of the BFS vial to secure the BFS vial to the delivery module.

40. The pre-filled delivery assembly of claim 37 wherein the administration member includes an administration hub, the administration hub coupled to the main body of the delivery module, the administration hub defining at least part of a mixing chamber for receiving the first and second fluid agents for delivery through the administration member.

41. The pre-filled delivery assembly of claim 40 wherein the main module includes a hub connector for coupling to the administration hub.

42. The pre-filled delivery assembly of claim 41 wherein the first and second internal penetrating members are integrally formed with the hub connector.

43. The pre-filled delivery assembly of claim 37 wherein the first and second internal penetrating members are staggered whereby the first internal penetrating member and the second internal penetrating member penetrate the respective first and second fluid seals in succession.

44. The pre-filled delivery assembly of claim 43 wherein the first internal penetrating member defines a length greater than a corresponding length of the second internal penetrating member.

45. The pre-filled delivery assembly of claim 37 including a removable protective cover disposed over the administration member.

46. The pre-filled delivery assembly of claim 37 including a pivoting shield pivotally mounted relative to the main body of the delivery module, the pivoting shield pivotal to selectively cover and expose the administration member.
